(19) United States
(12) Patent Application Publication
(10) Pub. No.: US 2024/0371466 A1
(43) Pub. Date: Nov. 7, 2024

(54) METHOD AND SYSTEM FOR NEWBORN SCREENING FOR GENETIC DISEASES BY WHOLE GENOME SEQUENCING

(71) Applicant: Rady Children's Hospital Research Center, San Diego, CA (US)

(72) Inventor: Stephen Kingsmore, San Diego, CA (US)

(21) Appl. No.: 18/291,186

(22) PCT Filed: Aug. 3, 2022
(86) PCT No.: PCT/US2022/039312

§ 371 Date: Jan. 22, 2024

Related U.S. Application Data
(60) Provisional application No. 63/229,460, filed on Aug. 4, 2021.

(51) Int. Cl. G16B 20/20; G16H 15/00; G16H 20/10; G16H 50/20

(52) U.S. Cl. CPC G16B 20/20; G16H 15/00; G16H 20/10; G16H 50/20

(57) ABSTRACT

The present disclosure provides a method and system for testing newborns for genetic diseases, diagnoses and implementing optimal treatments. The invention provides for rapid detection of genetic disease in newborns, as well as identification of available therapeutic interventions that may be rapidly implemented to prevent death or adverse complications characterized by the genetic disease.

### CROSS-REFERENCE TO RELATED APPLICATION(S)

This application claims benefit of priority under 35 U.S.C. § 119 (e) of U.S. Provisional Patent Application Ser. No. 63/229,460, filed Aug. 4, 2021. The disclosure of the prior application is considered part of and is incorporated by reference in the disclosure of this application.

### BACKGROUND OF THE INVENTION

### Field of the Invention

The invention relates generally to early targeted or precision treatment of genetic disease and more specifically to a method and system for screening all newborns for all genetic diseases that either have an effective treatment or that are amenable to development of a genetic therapy in order to implement optimal, etiology-informed management at or before onset of symptoms.

### Background Information

Newborn screening (NBS) is performed worldwide in ˜140 million newborns annually to identify severe congenital disorders and initiate treatments at or before onset of symptoms. While NBS can greatly improve health outcomes, the number of genetic disorders screened has not kept pace with genomic or therapeutic innovation. Between 2006 and 2022, the number of core disorders that were recommended for NBS of dried blood spots (DBS) in the United States—the Recommended Uniform Screening Panel (RUSP)—increased from 27 to 35, and the number of affected infants identified increased from 6,439 to 6,466. However, there are ˜7,200 known genetic diseases and hundreds of new targeted treatments that have been approved or are in clinical trials. Over the past decade, rapid WGS (rWGS®) has developed into an effective diagnostic test (Dx-rWGS®) for almost all heritable diseases and is gaining acceptance as a first-tier test for critically ill newborns with suspected genetic diseases. rWGS® is attractive for comprehensive NBS since it concomitantly examines almost all genetic diseases with similar time to result as biochemical NBS of DBS by mass spectrometry (NBS-MS).

More advanced methods are needed for automated screening of all newborns for all rare genetic diseases that either have an effective treatment or that are amenable to development of a genetic therapy in order to implement optimal, etiology-informed management at or before onset of symptoms, as described herein.

### SUMMARY OF THE INVENTION

The present invention provides a method and autonomous system for conducting genetic analysis of all rare genetic diseases that either have an effective treatment or that are amenable to development of a genetic therapy. The invention provides for rapid screening of genetic disease in all newborns.

Accordingly, in one embodiment the invention provides a method for conducting genetic analysis. The method includes:
a) determining a comprehensive set of genetic diseases that either have an effective treatment or that are amenable to development of a genetic therapy in a timeframe relevant to disease progression;
b) determining a set of genetic variants that are known to be pathogenic or likely pathogenic in the genes that map to that set of genetic diseases;
c) determining a subset of those genetic variants that have population allele frequencies (or diplotype allele frequencies) that are less than the incidence of the corresponding genetic diseases;
d) determining management guidelines regarding effective treatments or novel genetic therapy candidates for the set of diseases;
e) performing genetic sequencing of a DNA sample from the subject;
f) determining genetic variants of the DNA;
g) analyzing the results of (c) and (f) to generate a list of positive screening results;
h) recalculating the population allele frequencies (or diplotype allele frequencies) to include results of (f);
i) confirmatory testing of the results of (g) to determine whether they are true or false positives;
j) if the results of (i) are true positives, implementing the appropriate management guidelines of (d); and
k) updating the variant pathogenicity assertions of (b) to include results of (i).

In some aspects, the method further includes: l) determining the availability of confirmatory tests for the variants of (c).

In aspects, the method further includes identifying any clinical phenotypes of the subject prior (i) confirmatory testing by diagnostic interpretation of the positive screening results of (g). In certain aspects, translating the clinical phenotypes into a standardized vocabulary is performed by extraction of phenotypes from the electronic medical record by clinical natural language processing (CNLP) and then translation into one or more standardized vocabularies. In some aspects, genetic sequencing includes rWGS®, rapid whole exome sequencing (rWES), or rapid gene panel sequencing.

The present invention further provides a method and autonomous system for conducting genetic analysis at population scale. The invention provides newborn screening for early diagnosis and treatment of genetic disease.

In one embodiment the invention provides a method for conducting genetic analysis. The method includes:
a) determining a comprehensive set of genetic diseases;
b) identifying genetic diseases of the comprehensive set that are severe and have childhood onset;
c) determining efficacy and quality of evidence of efficacy of a comprehensive set of available therapeutic interventions for the genetic disease identified in (b);
d) determining a comprehensive set of genes associated with genetic diseases that have at least one available therapeutic intervention;
e) determining a comprehensive set of pathogenic or likely pathogenic genetic variants of the comprehensive set of genes determined in (d);
f) determining population frequency of the genetic variants;
g) for recessive genetic diseases of the genetic variants, determining which recessive genetic diseases occur in cis in populations;
h) analyzing results of (e), (f) and (g) to generate a revised list of pathogenic or likely pathogenic genetic variants;
i) performing genetic sequencing of a genomic DNA sample from a subject;
j) determining genetic variant diplotypes of the genomic DNA;
k) comparing the genetic variant diplotypes with the results of (h) to determine whether the subject screens positive for a genetic disease for which an effective treatment currently exists or can be developed; and
l) generating a report including results of any of (a)-(k).

In another embodiment the method includes:
a) determining a comprehensive set of disease-causing genes;
b) determining a comprehensive set of pathogenic or likely pathogenic variants in disease-causing genes;
c) determining the subset of those variants for which an effective genetic therapy can be developed;
d) determining the efficacy and/or quality of evidence of efficacy of available treatments for the set of disease-causing genes;
e) analyzing the results of (b), (c) and (d) to generate a list of pathogenic or likely pathogenic variants in disease-causing genes for which an effective therapy is available or are amenable to development of an effective genetic therapy;
f) performing genetic sequencing of a genomic DNA sample from a subject;
g) determining genetic variant diplotypes of the genomic DNA;
h) comparing the genetic variant diplotypes of the subject with the results of (b) and (c) to determine whether the subject has a genetic disease for which an effective treatment currently exists or can be developed; and
i) generating a report including results of any of (a)-(h).

In another embodiment, the invention provides a system for performing a method of the invention. The system includes a controller having at least one processor and non-transitory memory. The controller is configured to perform one or more of the processes of the method as described herein.

### BRIEF DESCRIPTION OF THE DRAWINGS

FIGS. 1A-1B depicts flow diagrams of the diagnosis of genetic diseases by standard and rapid genome sequencing. FIG. 1A is a flow diagram of the diagnosis of genetic diseases. FIG. 1B is a flow diagram of the diagnosis of genetic diseases.

FIGS. 2A-2B depicts diagrams showing clinical natural language processing can extract a more detailed phenome than manual electronic health record (EHR) review or Online Mendelian Inheritance in Man™ (OMIM™) clinical synopsis. FIG. 2A is a schematic diagram. FIG. 2B is a schematic diagram.

FIGS. 3A-3H depicts a comparison of observed and expected phenotypic features of children with suspected genetic diseases. FIG. 3A is a graphical diagram depicting data. FIG. 3B is a graphical diagram depicting data. FIG. 3C is a graphical diagram depicting data. FIG. 3D is a Venn diagram depicting data. FIG. 3E is a graphical diagram depicting data. FIG. 3F is a graphical diagram depicting data. FIG. 3G is a graphical diagram depicting data. FIG. 3H is a Venn diagram depicting data.

FIG. 4 is a Venn diagram showing overlap of observed and expected patient phenotypic features in 95 children diagnosed with 97 genetic diseases.

FIGS. 5A-5B are a series of graphs depicting precision, recall, and F1-score of phenotypic features identified manually, by CNLP, and OMIM™. FIG. 5A is a series of graphical diagrams depicting data. FIG. 5B is a series of graphical diagrams depicting data.

FIG. 6 is a flow diagram illustrating the software components of the autonomous system and methodology for provisional diagnosis of genetic diseases by rapid genome sequencing in one aspect of the invention.

FIG. 7 is a flow diagram illustrating the software components of the autonomous system and methodology for provisional diagnosis of genetic diseases by rapid genome sequencing in one aspect of the invention.

FIGS. 8A-8B are flow diagrams of the technological components of a 13.5-hour system for automated diagnosis and virtual acute management guidance of genetic diseases by rWGS® in an aspect of the invention. FIG. 8A is a flow diagram showing the order and duration of laboratory steps and technologies. FIG. 8B is a flow diagram showing the information flow from order placement in the EHR to return of diagnostic results together with specific management guidance for that genetic disease.

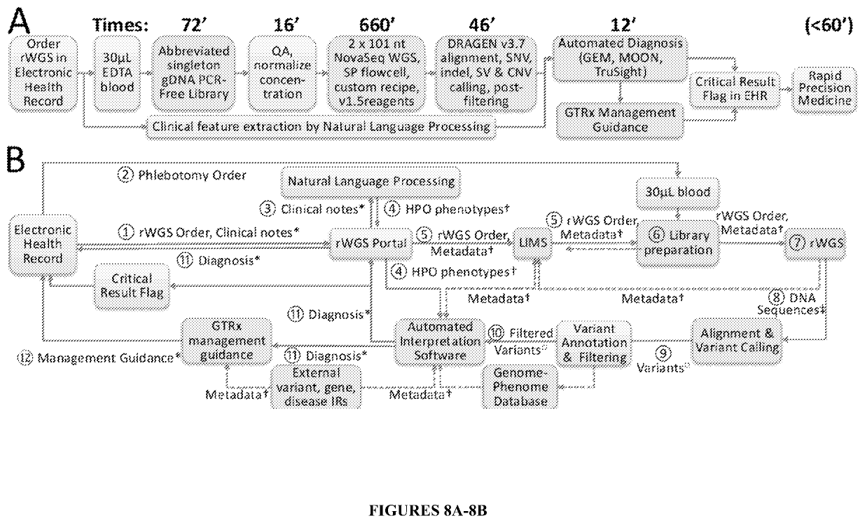

FIG. 9 is a flow diagram illustrating the development of Genome-To-Treatment (GTRx℠), a virtual system for acute management guidance for rare genetic diseases.

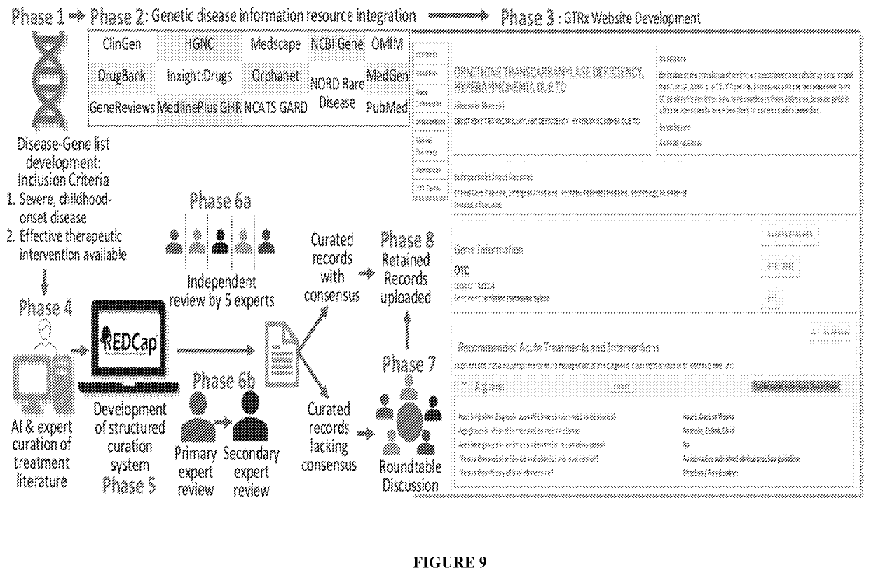

FIGS. 10A-10B illustrates GTRx℠ disease, gene, and literature filtering, and final content. FIG. 10A is a modified PRISMA flowchart showing filtering steps and summarizing results of review of 563 unique disease-gene dyads herein. FIG. 10B is a diagram showing genetic disease types and disease genes featured in the first 100 GTRx℠ genes reviewed herein.

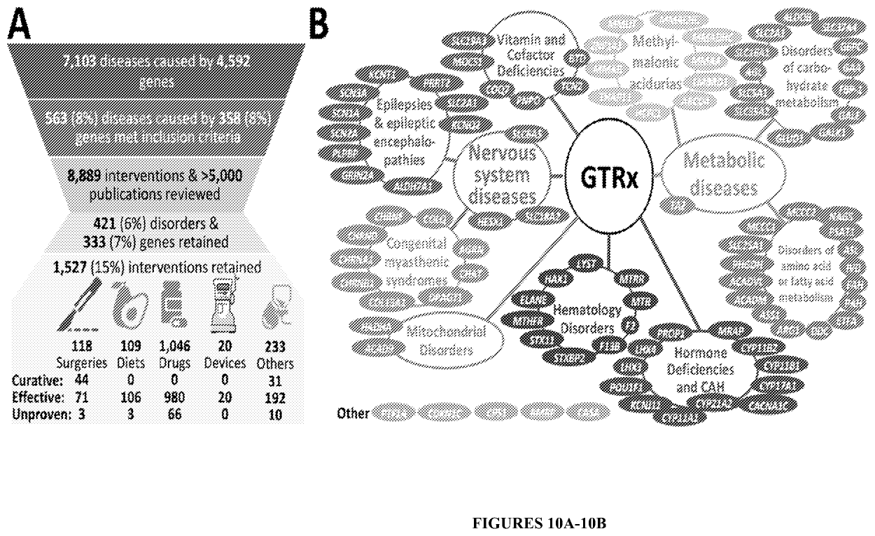

FIGS. 11A-11D depicts data derived using the system and methodology of the present invention. FIG. 11A shows clinical timeline of a patient. FIG. 11B shows diagnostic timeline of a patient. FIG. 11C shows clinical timeline of a patient. FIG. 11D shows diagnostic timeline of a patient.

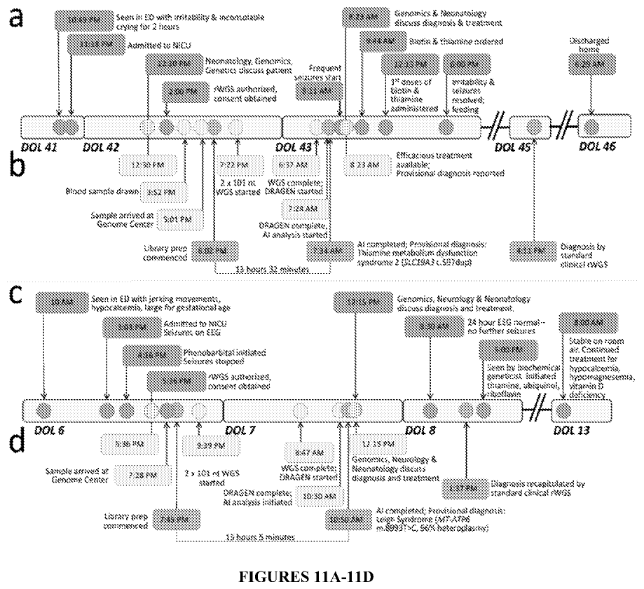

FIG. 12 is a graphical plot depicting data pertaining to genetic sequencing costs.

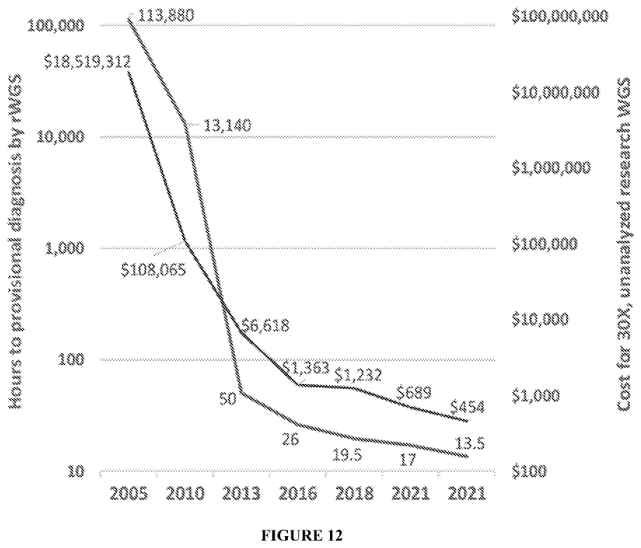

FIG. 13 is a flowchart showing the modified Delphi technique for ongoing selection of disorders for NBS-rWGS® after they have been included in the GTRx℠ virtual management guidance system GTRx℠.

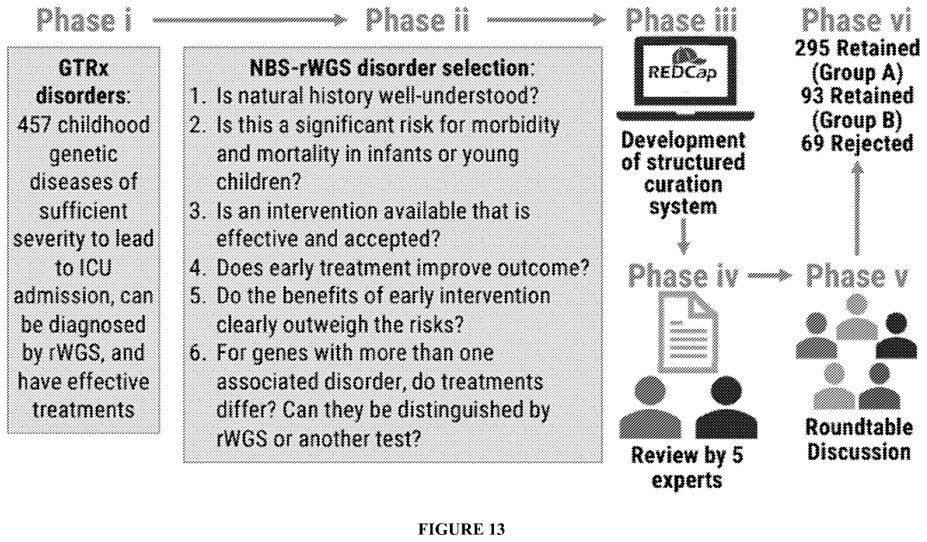

FIGS. 14A-14C show a comparison of the workflow for Dx-rWGS®. FIG. 14A is a comparison for NBS-rWGS®. FIG. 14B is a comparison for a secondary use of data generated by NBS-rWGS®. FIG. 14C illustrates that the interpretation burden of NBS-rWGS® is approximately 1,000-fold less than that of Dx-rWGS®. The light blue shading indicates the activities occurring in places of care for newborns or older children, while the darker blue sharing indicates activities occurring in clinical laboratories. The dashed green arrows \textcircled{1} and \textcircled{2} in NBS-rWGS® indicate feedback loops. Abbreviations: dB, database; EDTA, ethylene diamine tetra-acetic acid; ICU, intensive care unit; EHR, electronic health record; CLIA, clinical laboratory improvements act; GEM™ AI, a genome interpretation tool that employs artificial intelligence¹⁵; GTRx℠, Genome-to-Treatment virtual management guidance system.

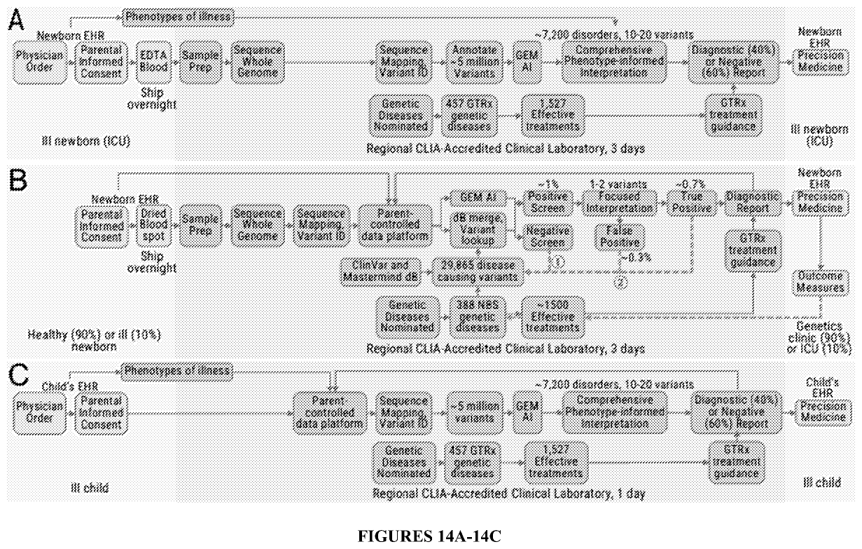

FIGS. 15A-15B are funnel plots. FIG. 15A shows reduction in 2,982 positive individuals in 73 positive NBS-rWGS® genes among 454,707 UK Biobank participants by root cause analysis. FIG. 15B shows the increase in retrospective NBS-rWGS® positives among 4,376 children and their parents. Abbreviations: LB, likely benign; B, benign; AR, autosomal recessive; AD, autosomal dominant; ICD, International Statistical Classification of Diseases and Related Health Problems; dB, database; UKBB, United Kingdom Biobank.

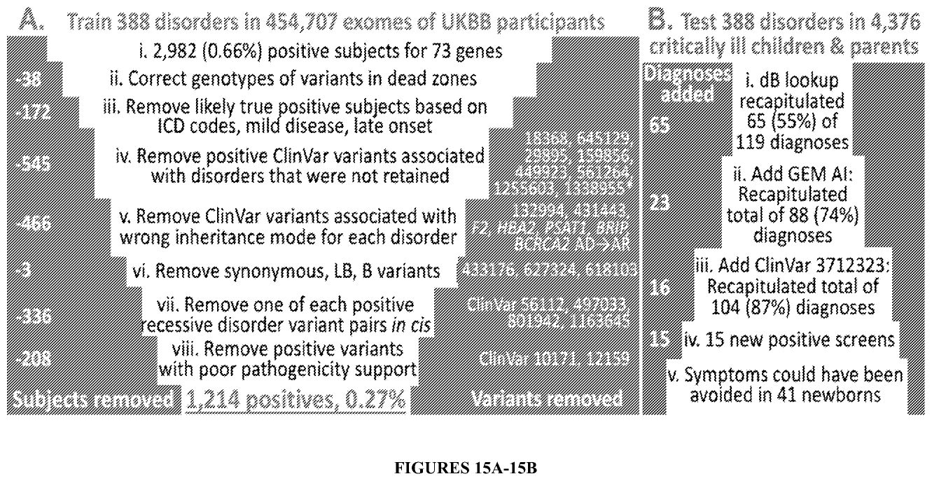

FIGS. 16A-16C depict the impact of training on the sensitivity and specificity of NBS-MS and NBS-rWGS®. FIG. 16A illustrates use of postanalytical tools to reduce false positives from NBS-MS of 48 disorders from 454 to 41, improving specificity (true negative rate) from 99.7% to 99.98%. Of note, false positives excluded newborns with birth weight <1.8 kg and DBS obtained at <24 hours or >7 days. FIG. 16B illustrates use of root cause analysis to reduce NBS-false positives from NBS-rWGS® of 388 disorders from 2,982 to 1,214, improving specificity from 99.3% to 99.7%. FIG. 16C shows that addition of positive individuals by GEM™ and inclusion of ClinVar™ 3712323 increased NBS-rWGS® true positives from 65 to 104, improving sensitivity from 59.6% to 87%. Of note, these results included NBS-rWGS® of newborns with birth weight <1.8 kg and DBS obtained at >7 days.

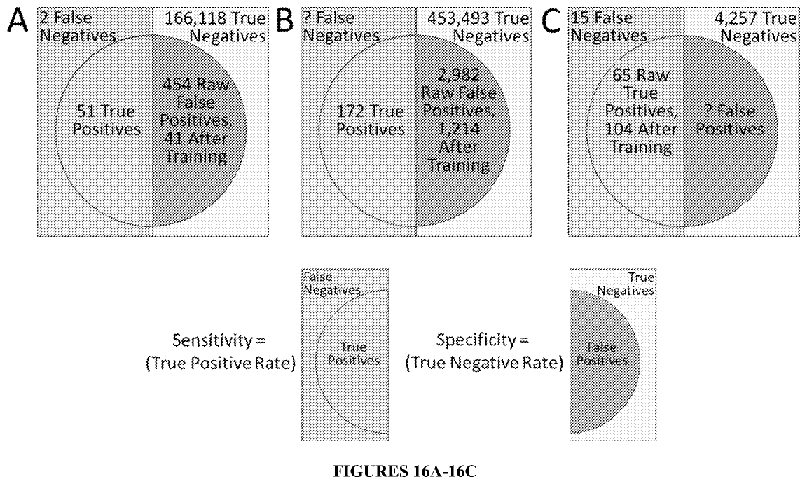

FIG. 17 is a visualization of paired sequence reads on a 120 nt region of Chr 1 demonstrating that ClinVar™ variants 280113 (PKLR g.155,294,726G>T, p.Glu241Ter), shown in green, and 1163645 (PKLR g.155294621del, p. Val276fs), shown as a black hash, occurred in the same read in a positive UKBB subject (boxes).

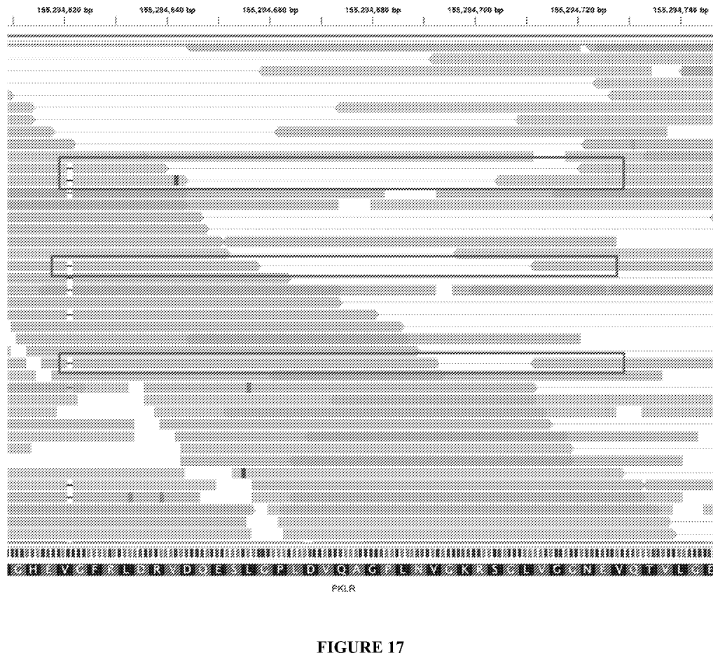

### DETAILED DESCRIPTION OF THE INVENTION

The present invention is based on an innovative computational method and platform for genomic analysis.

Herein the inventors describe an innovative, scalable solution to Scylla and Charybdis of diagnostic and therapeutic odysseys in rapidly progressive childhood genetic diseases. Firstly, the inventors describe automated platform for rWGS® in 13.5 hours that allows even the most rapidly progressive genetic diseases to be therapeutically tractable. Secondly, rather than ending rWGS® with static molecular results, the inventors describe methods for dynamic reports that extend to integrated information resources and optimized, acute management guidance designed for front-line, intensive care physicians.

Accordingly, the disclosure describes scalable, feedback-informed methods for newborn screening, diagnosis, and virtual, acute management guidance for 388 diseases, and reports analytic performance and clinical utility in large retrospective datasets.

As discussed in detail in the Examples, by informing timely targeted treatments, rapid genetic or genomic sequencing can improve the outcomes of seriously ill children with genetic diseases, particularly infants in neonatal and pediatric intensive care units (ICUs). The need for highly qualified professionals to decipher results, however, precludes widespread implementation.

In various aspects, the present disclosure provides a platform for population-scale, provisional diagnosis of genetic diseases with automated phenotyping and interpretation. Many rare genetic diseases with effective treatments progress to severe morbidity or mortality if untreated immediately. Front-line physicians are often unfamiliar with treatments for these diseases. Hence rapid molecular diagnosis may be insufficient to improve outcomes. The inventors describe Genome-to-Treatment (GTRx℠), an automated system for genetic disease diagnosis and acute management support. Diagnosis was achieved in 13.5 hours by sequencing library preparation directly from blood, accelerated whole genome sequencing (WGS), hyperthreaded informatic analysis, natural language processing of electronic health records and automated interpretation. 563 severe, childhood-onset, genetic diseases with effective treatments were identified by literature review, clinician nomination and WGS experience. Specific treatments, including drugs, devices, diets, and surgeries, were identified for each by internet and literature searches, and manually curated. Five clinical geneticists adjudicated the indications, contraindications, efficacy, and evidence-of-efficacy of each treatment in each disorder in the context of a newly diagnosed, ill child in an intensive care unit (ICU). After discussion, they agreed upon 189 of the first 190 treatments. The inventors integrated 10 genetic disease information resources, and electronically linked them and the adjudicated treatments to automated diagnostic reports (rbsapp.net:8082). The 13.5-hour system had superior analytic performance for single nucleotide, insertion-deletion, structural and copy number variants. GTRx℠ provided correct diagnoses and management guidance in four retrospective patients. Prospectively, an infant with encephalopathy was diagnosed in 13.5 hours, enabling timely institution of effective treatment. GTRx℠ facilitates prompt diagnosis and implementation of optimized, acute treatment for patients with rapidly progressive genetic diseases, particularly in ICUs staffed by front-line physicians.

In various embodiments, the disclosure describes adaptation of Dx-rWGS® methods for comprehensive NBS (NBS-rWGS®).

Before the present compositions and methods are described, it is to be understood that this invention is not limited to particular methods and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

An initial, general solution to the problem of rapidly progressive childhood genetic diseases was rapid diagnostic WGS. Rapid WGS mitigated the problem of unknown etiology, wherein it was impossible to make a molecular diagnosis for most genetic diseases during hospitalization. Since then, rapid WGS has increased in speed, diagnostic performance, and scalability. Rapid WGS now allows concomitant evaluation of almost all differential diagnoses—which may number over 1,000 genetic disorders in a single patient. Rapid WGS has started to be implemented nationally for inpatient diagnosis of genetic disease in England, Australia, and Wales and in several US states.

As is often true of new technologies, rapid WGS removed the rare disease diagnostic odyssey bottleneck, but exposed another downstream bottleneck—the therapeutic odyssey that results in missed opportunity for clinical. Clinical trials of rapid WGS have repeatedly shown gaps between expected and observed clinical utility. Several factors contribute to missed clinical utility. Firstly, exponential advances in genomics have outpaced medical education. Many healthcare providers lack adequate genomic literacy to practice genomic medicine unaided. Neonatologists, intensivists, and hospitalists are often dependent upon other subspecialists, particularly medical geneticists, for translation of rapid WGS results into treatment recommendations. In quaternary hospitals, this leads to treatment delays. In front-line settings, however, it can greatly limit the clinical utility of rapid WGS. Secondly, many genetic diseases were either discovered only recently, or are ultra-rare, and therefore evidence-based treatment guidelines have not yet been developed. Moreover, effective management strategies are often interspersed across the literature in the form of case reports, case series or small cohort studies. Information resources pertaining to management of rare genetic diseases are incomplete, lack interoperability, and are typically not targeted toward acute ICU treatment or front-line physicians. Upon receipt of a rapid WGS-based diagnosis, these factors put an unsupportable burden on front-line physicians to search and synthesize the available evidence for rare genetic diseases, many of which they may have never encountered previously. Therapeutic unfamiliarity will continue to increase as new diseases and new effective, n-of-few, genetic therapies proliferate. Thirdly, failure to order rapid WGS as a first-tier test frequently leads to return of results around time of hospital discharge, when management plans have been solidified or, for rapidly progressive diseases, too late to have full clinical utility.

While historically it took an average of twelve years for drug development and approval, effective genetic therapies for genetic diseases can be developed and receive expanded-access investigational clinical protocol authorization by the Food and Drug Administration in as little as one year (such as milasen for Batten disease); Thus, newborn screening by WGS should consider not only those conditions for which current treatments exist, but also conditions for which novel genetic therapies can be developed in a timeframe that is pertinent to disease progression. 2. Genetic therapies can delay progression and death in patients with fatal genetic diseases (such as onasemnogene abeparvovec for the treatment of symptomatic patients with spinal muscular atrophy and eteplirsen for Duchenne Muscular Dystrophy. However, these genetic therapies do not reverse damage to organs. Instead, they prevent disease progression. Thus, it is necessary to identify these conditions at birth, rather than at onset of symptoms in order to have maximal efficacy. 3. While we now know the cause of more than 6,100 genetic diseases, for most genomic diagnoses, frontline physicians will never have encountered a patient with that disorder, indicating the need to provide management guidance as part of a system of newborn screening by WGS. 4. While effective treatments exist for ˜600 genetic diseases, for the vast majority the evidence of effectiveness is limited to case reports and case series, preventing ready access to such knowledge by frontline physicians, further indicating the need to provide management guidance as part of a system of newborn screening by WGS. 5. Traditional, population newborn screening has been highly effective but is currently limited to 35 core conditions on the Advisory Committee on Heritable Disorders in Newborns and Children's Recommended Uniform Screening Panel. As a result, there remain hundreds of genetic diseases with effective treatments that are not currently screened for. 6. The cost of research-quality human genome sequencing has dropped to $689 and is projected to be $100 in a few years. 7. California Newborn screening currently costs $129 per subject, and therefore genome sequencing is approaching the cost effectiveness needed for newborn screening. 8. Genome sequencing is now possible in 13.5 hours, a turnaround time that is sufficient for newborn screening; 9. Genome sequence analysis can be completely automated and therefore scaled to populations, as needed for newborn screening of approximately 4 million US births per year. 10. Genome sequencing, analysis and virtual treatment guidance can be completely automated, which is necessary for these methods to be scalable to populations.

Traditional newborn screening is focused on screening for a small number of individual diseases for which strong evidence supports the effectiveness of currently available treatments. Previous attempts to convert newborn screening to whole exome sequencing have also focused on screening for the same types of diseases. They have failed because of insufficient sensitivity relative to traditional newborn screening. For example, newborn screening of 48 disorders by whole exome sequencing had a sensitivity of 88% compared to 99.0% for traditional newborn screening. In another example, newborn screening by whole exome sequencing had a sensitivity of 88% in children with metabolic disorders and 18% in children with hearing loss. The system of newborn screening by WGS disclosed herein instead focuses on screening all ˜600 genetic diseases with effective treatments and all genetic diseases for which novel genetic therapies can be developed in a timeframe that is pertinent to disease progression. It should be noted that novel genetic therapies are often designed based not on the disorder pathology but rather on the class of genetic variant that causes the condition. Thus, patients with any disorder that is caused by variants that create premature stop codons may potentially be effectively treated with antisense allele specific oligonucleotide therapies that alter exon skipping. Newborn screening by WGS if focused on tens of thousands of variant diplotypes that are known to be pathogenic or known and likely to be pathogenic (defined by a subset of the American College of Medical Genetics criteria) that map to all ˜600 genetic diseases with effective treatments and all genetic diseases for which novel genetic therapies can be developed in a timeframe that is pertinent to disease progression. Thus, newborn screening by WGS achieves cost effectiveness and clinical utility in aggregate across tens of thousands of variants and hundreds screened conditions, rather than on a condition-by-condition basis. Insensitivity for any single screened variant or condition (“missing” true positives) is acceptable provided the aggregate clinical utility and cost effectiveness across all conditions and variants is acceptable. This is because the incremental cost of adding a new condition or variant to newborn screening by WGS is negligible, whereas in traditional newborn screening it was substantial.

Genome sequencing results in identification of 6 million variants per subject, most of which do not cause disease. Previous attempts to convert newborn screening to whole exome sequencing have utilized conventional interpretation methods that were frustrated by the need for many hours of interpretation and many false positives (low precision). For example, newborn screening of 48 disorders by whole exome sequencing had specificity of 98.4%, compared to 99.8% for traditional newborn screening. For population newborn screening to be effective it must have an extremely low rate of false positives (high precision). By focusing on tens of thousands of variant diplotypes that are known to be pathogenic or known and likely to be pathogenic (defined by a subset of the American College of Medical Genetics criteria), and that have population frequencies that are less than that of the condition being tested for, newborn screening by WGS system achieves the requisite precision for population implementation.

Conventional diagnostic WGS takes skilled operators at least six hours to analyze and interpret manually; the methods for newborns screening by WGS disclosed herein take less than one minute to execute computationally with no human effort.

Traditional newborn screening is designed for a rather static set of disorders, with at best annual changes in screened disorders, decided upon by a federal committee. Likewise, previous attempts to convert newborn screening to whole exome sequencing and panel tests have been static. In addition, neither traditional screening nor previous attempts to convert newborn screening to whole exome sequencing and panel tests include disorders for which novel genetic therapies could be developed in a meaningful timeframe. The methods of newborn screening by WGS disclosed herein are highly dynamic-conditions and variants can be added or subtracted even nightly.

Previous attempts to convert newborn screening to whole exome sequencing or panel tests used static variant pathogenicity assertions and were not designed to be self-learning. With few exceptions, sensitivity and specificity were not improved based on learning from tested subjects. The methods of newborn screening by WGS disclosed herein were designed to be self-learning: Each individual patient's set of variants are uploaded into a master database of variants and the allele frequency of each variant in the entire tested population is recalculated nightly. Thus, likely false positive variants that occur more frequently in the population than the incidence of the corresponding genetic disease can be identified and blocked from consideration in subsequent patients tested. Likewise, upon confirmatory testing, true positive and false positive results are uploaded into the master database and the pathogenicity assertion is updated for all subsequent patients tested. Self-learning cannot be dynamically retrofitted to a conventional, dense array database in which each patient adds 6 billion null values and six million non-null values. Instead, a non-obvious, sparse array database solution is needed that features exceptionally fast read/write capability and that is designed to support self-learning with regard to variant frequency and confirmatory test results. The database solution disclosed herein features sparse array representation of only the six million non-null WGS variants and of the ˜30,000 variants that are screened for that is optimized for exceptionally fast read/write capability and designed to support self-learning with regard to variant frequency and confirmatory test results on a per subject basis. The attributes of data storage managers sufficient for screening of millions of newborns per year for hundreds of genetic diseases by WGS.

Previous attempts to convert newborn screening to whole exome sequencing or panel tests were predicated upon selection of disorders that were “actionable,” meaning likely to result in a change in clinical management of the subject. As noted above, effective management strategies for individual genetic diseases are often interspersed across the literature in the form of case reports, case series or small cohort studies. It is thus non-obvious which disorders should be included in newborn screening by WGS. In the methods disclosed herein, inclusion of disorders and variants were predicated on expert curation of the subset of those variants for which an effective genetic therapy can be developed and the efficacy and/or quality of evidence of efficacy of available treatments for the set of disease-causing genes.

### Methods

In one embodiment, the invention provides a method for conducting genetic analysis at population scale for newborns. The invention provides for early diagnosis and treatment of genetic disease, for example in a fetus, neonate or infant.

In some aspects, the method includes:
a) determining a comprehensive set of genetic diseases;
b) identifying genetic diseases of the comprehensive set that are severe and have childhood onset;
c) determining efficacy and quality of evidence of efficacy of a comprehensive set of available therapeutic interventions for the genetic disease identified in (b);
d) determining a comprehensive set of genes associated with genetic diseases that have at least one available therapeutic intervention;
e) determining a comprehensive set of pathogenic or likely pathogenic genetic variants of the comprehensive set of genes determined in (d);
f) determining population frequency of the genetic variants;
g) for recessive genetic diseases of the genetic variants, determining which recessive genetic diseases occur in cis in populations;
h) analyzing results of (e), (f) and (g) to generate a revised list of pathogenic or likely pathogenic genetic variants;
i) performing genetic sequencing of a genomic DNA sample from a subject;
j) determining genetic variant diplotypes of the genomic DNA;
k) comparing the genetic variant diplotypes with the results of (h) to determine whether the subject screens positive for a genetic disease for which an effective treatment currently exists or can be developed; and
l) generating a report including results of any of (a)-(k).

In some aspects, the method includes:
a) determining a comprehensive set of disease-causing genes;
b) determining a comprehensive set of pathogenic or likely pathogenic variants in disease-causing genes;
c) determining the subset of those variants for which an effective genetic therapy can be developed;
d) determining the efficacy and/or quality of evidence of efficacy of available treatments for the set of disease-causing genes;
e) analyzing the results of (b), (c) and (d) to generate a list of pathogenic or likely pathogenic variants in disease-causing genes for which an effective therapy is available or are amenable to development of an effective genetic therapy;
f) performing genetic sequencing of a genomic DNA sample from a subject;
g) determining genetic variant diplotypes of the genomic DNA;
h) comparing the genetic variant diplotypes of the subject with the results of (b) and (c) to determine whether the subject has a genetic disease for which an effective treatment currently exists or can be developed; and
i) generating a report including results of any of (a)-(h).

In one embodiment the invention provides a method for conducting genetic analysis. The analysis may be utilized to diagnose a disease or disorder, in particular a rare genetic disease. The method can also be utilized to rule out a genetic disease. The method of the invention is particularly useful in detecting and/or diagnosing a genetic disease in a subject that is less than 5 years old, such as an infant, neonate or fetus.

In some aspects, the method includes:
a) determining a phenome of a subject from an electronic medical record (EMR), wherein the phenome includes a plurality of clinical phenotypes extracted from the EMR;
b) translating the clinical phenotypes into standardized vocabulary or vocabularies;
c) generating a first list of potential differential diagnoses of the subject;
d) performing genetic sequencing of a DNA sample from the subject;
e) determining genetic variants of the DNA;
f) analyzing the results of (c) and (e) to generate a second list of potential differential diagnoses of the subject, the second list being rank ordered;
g) determining the efficacy and/or quality of evidence of efficacy of available treatments for the second list of potential differential diagnoses;
h) analyzing the results of (f) and (g) to generate a third list of potential differential diagnoses of the subject, the third list being rank ordered, together with available treatments; and
k) generating a report including results of any of (a)-(h).

In some aspects, the method further includes: j) determining the availability of confirmatory tests for the third list of potential differential diagnoses.

In some aspects, the method further includes: k) analyzing the results of (g) and (h) to generate a fourth list of potential differential diagnoses of the subject, the fourth list being rank ordered, together with available confirmatory tests.

In some aspects, the method may further include generating the EMR for the subject prior to determining the phenome of the subject.

As used herein, “phenome” refers to the set of all phenotypes expressed by a cell, tissue, organ, organism, or species. The phenome represents an organisms' phenotypic traits.

As used herein, “EMR” refers to an electronic medical record and is used synonymously herein with “electronic health record” or “EHR”.

The method includes determining a phenome of a subject from an electronic medical record (EMR). This is performed by extracting a plurality of clinical phenotypes from the EMR. Natural language processing and/or automated feature extraction from non-standardized and standardized fields of the EMR of a subject is used to create a list of the clinical features of disease in that individual.

Translating the clinical phenotypes into standardized vocabulary is then performed utilizing a variety of computation methods known in the art. In one aspect, translation is performed by natural language processing. This type of processing is utilized for translation and mining of non-structured text. Alternatively, data organized in discrete or structured fields may be retrieved/translated utilizing a conventional query language known in the art. Embodiments of standardized vocabularies include the Human Phenotype Ontology, Systematized Nomenclature of Medicine—Clinical Terms, and International Classification of Diseases—Clinical Modification.

The method also entails generating a series of lists (e.g., first, second, third, fourth, and the like) of potential differential diagnoses of the subject. In some aspects, the method entails generating a first list of potential differential diagnoses. This is performed by query of a database populated with known clinical phenotypes expressed in the same vocabulary as the standardized vocabulary of the translated clinical phenotypes. Embodiments of databases of known clinical phenotypes include Online Mendelian Inheritance in Man-Clinical Synopsis, and Orphanet Clinical Signs and Symptoms. The list may be generated with an algorithm that rank orders all potential differential diagnoses based on goodness of fit. The list may also be generated with an algorithm that rank orders all potential differential diagnoses based on the sum of the distances of the observed and expected phenotypes in the standardized, hierarchical vocabulary.

Genetic variants are then determined from genomic sequencing performed on a DNA sample from the subject. In some aspects, this includes annotation and classification of the genetic variants. Annotation of all, or some, of the genetic variations in the subject's genome is performed to identify all variants that are of categories such as uncertain significance (VUS), pathogenic (P) or likely pathogenic (LP) and to retain genetic variations with an allele frequency of <5, 4, 3, 2, 1, 0.5, or 0.1% in a population of healthy individuals. The method may further include annotation of the genetic variants to identify and rank all diplotypes categorically, for example as being of uncertain significance (VUS), pathogenic (P) or likely pathogenic (LP) on the basis of pathogenicity. An embodiment of the classification system is the Joint Consensus Recommendation of the American College of Medical Genetics and Genomics and the Association for Molecular Pathology Standards and Guidelines for the Interpretation of Sequence Variants. The method may further include annotation of the pathogenicity of variants and diplotypes on a continuous, probabilistic scale, where a variant that is well established to be benign, for example, has a score of zero, and a variant that is well established to be pathogenic variant has a score of one, and likely benign, variants of uncertain significance, and likely pathogenic variants have scores between zero and one.

A second list of potential differential diagnoses of the subject is then generated by comparing the annotated VUS, LP and P diplotypes on a regional genomic basis with corresponding genomic regions associated with the first list of potential differential diagnoses. Genetic variants are ranked based on a combination of rank of goodness of fit of clinical phenotypes, rank of pathogenicity of diplotypes, and/or allele frequencies of the genetic variants in a population of healthy individuals. The list of potential differential diagnoses may further include annotation of their probability of being causative of the patient's condition on a continuous scale, rather than binary diagnosis/no diagnosis results.

In some aspects, the genetic variants determined from the subject's genome may be utilized to generate a probabilistic diagnosis for use in generating the second list of potential diagnoses.

A report is then generated setting forth the potential differential diagnoses of the subject, preferably in order of score to identify the diagnosis with the highest probability.

In some aspects, the method entails generating a third list, and optionally a fourth list of potential differential diagnoses. This is performed by query of a database populated with known clinical phenotypes expressed in the same vocabulary as the standardized vocabulary of the translated clinical phenotypes. Embodiments of databases of known clinical phenotypes include Online Mendelian Inheritance in Man-Clinical Synopsis, and Orphanet Clinical Signs and Symptoms. The lists may be generated with an algorithm that rank orders all potential differential diagnoses based on goodness of fit. The lists may also be generated with an algorithm that rank orders all potential differential diagnoses based on the sum of the distances of the observed and expected phenotypes in the standardized, hierarchical vocabulary.

In various aspects, the method includes determining the efficacy and/or quality of evidence of efficacy of available treatments for the list of potential differential diagnoses. In various aspects, the generated list of potential differential diagnoses of the subject, is rank order and accompanied by the suitable available treatments.

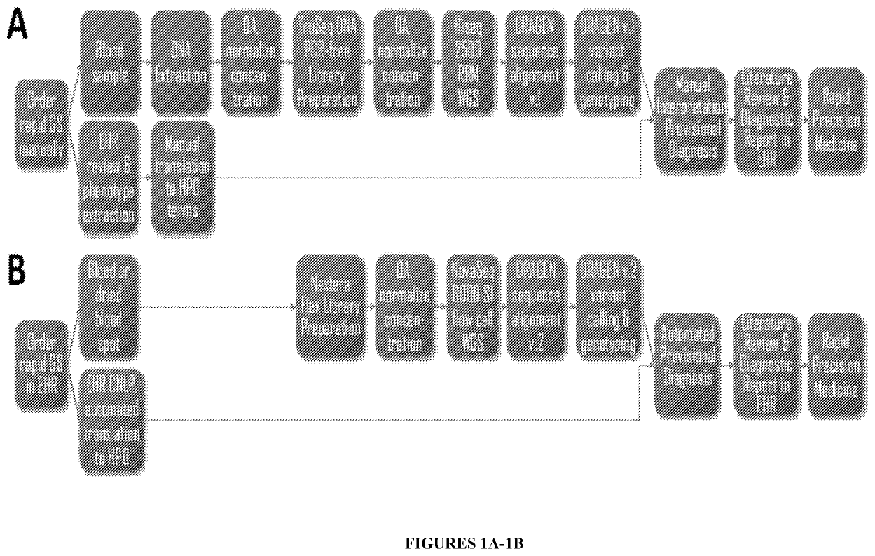

Some aspects of the invention are illustrated in FIG. 1B. FIG. 1B is a flow chart showing AI involved automated extraction of the phenome from subject's EMR by clinical natural language processing (CNLP), translation from SNOMED-CT to Human Phenotype Ontology (HPO) terms (e.g., a standardized vocabulary), derivation of a comprehensive differential diagnosis gene list, identification of variants in genomic sequences, assembling those variants into likely pathogenic, causal diplotypes on a gene-by-gene basis, integration of the genotype and differential diagnosis lists, and retention of the highest ranking provisional diagnosis(es).

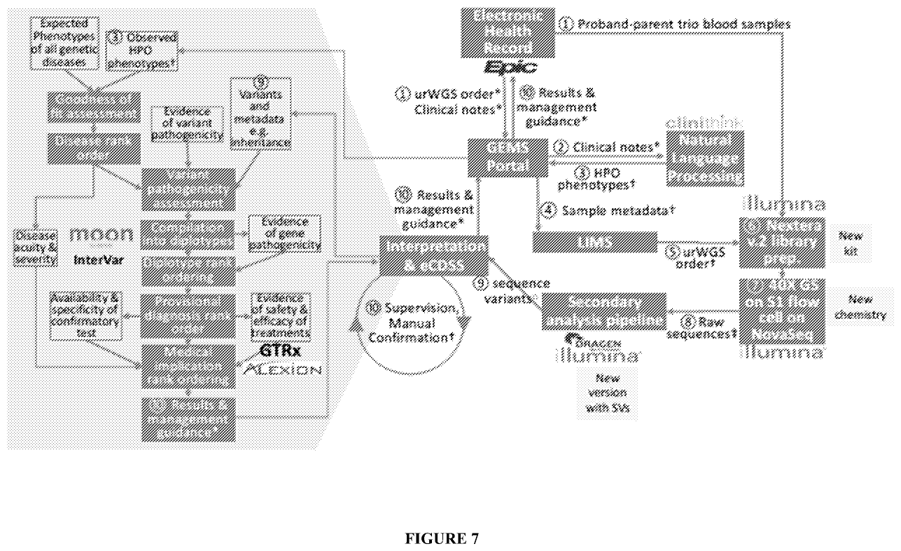

Some aspects of the invention are illustrated in FIG. 7 which is a flow diagram illustrating components of the autonomous system and methodology for diagnosis of genetic diseases by rapid genome sequencing.

The method of the present invention allows for a myriad of genetic analysis types to identify disease.

Methods described herein are useful in perinatal testing wherein the parental, e.g., maternal and/or paternal, genotypes are known. In an aspect, the methods are used to determine if a subject has inherited a deleterious combination of markers, e.g., mutations, from each parent putting the subject at risk for disease, e.g., Lesch-Nyhan syndrome. The disease may be an autosomal recessive disease, e.g., Spinal Muscular Atrophy. The disease may be X-linked, e.g., Fragile X syndrome. The disease may be a disease caused by a dominant mutation in a gene, e.g., Huntington's Disease. In some aspects, the maternal nucleic acid sequence is the reference sequence. In some aspects, the paternal nucleic acid sequence is the reference sequence. In some aspects, the marker(s), e.g., mutation(s), are common to each parent. In some aspects, the marker(s), e.g., mutation(s), are specific to one parent.

In some aspects, haplotypes of an individual, such as maternal haplotypes, paternal haplotypes, or fetal haplotypes are constructed. The haplotypes include alleles co-located on the same chromosome of the individual. The process is also known as “haplotype phasing” or “phasing”. A haplotype may be any combination of one or more closely linked alleles inherited as a unit. The haplotypes may include different combinations of genetic variants. Artifacts as small as a single nucleotide polymorphism pair can delineate a distinct haplotype. Alternatively, the results from several loci could be referred to as a haplotype. For example, a haplotype can be a set of SNPs on a single chromatid that is statistically associated to be likely to be inherited as a unit.

In some aspects, the maternal haplotype is used to distinguish between a fetal genetic variant and a maternal genetic variant, or to determine which of the two maternal chromosomal loci was inherited by the fetus.

In some aspects, the methods provided herein may be used to detect the presence or absence of a genetic variant in a region of interest in the genome of a subject, such as an infant or fetus in a pregnant woman, wherein the fetal genetic variant is an X-linked recessive genetic variant. X-linked recessive disorders arise more frequently in male fetus because males with the disorder are hemizygous for the particular genetic variant. Example X-linked recessive disorders that can be detected using the methods described herein include Duchenne muscular dystrophy, Becker's muscular dystrophy, X-linked agammaglobulinemia, hemophilia A, and hemophilia B. These X-linked recessive variants can be inherited variants or de novo variants.

In some aspects, provided herein is a method of detecting the presence or absence of a genetic variant in a region of interest in the genome of an infant or a fetus in a pregnant woman, wherein the fetal genetic variant is a de novo genetic variant or a maternally or paternally inherited genetic variant. In some aspects, the mother's and/or the father's genome is sequenced to reveal whether the genetic variant is a maternally or paternally inherited genetic variant or a de novo genetic variant. That is, if the fetal genetic variant is not present in the mother or the father, and the described method indicates that the fetal genetic variant is distinguishable from the maternal or the paternal genome, then the fetal genetic variant is a de novo variant. Accordingly, provided herein is a method of determining whether a fetal genetic variant is an inherited genetic variant or a de novo genetic variant.

In some aspects, provided herein is a method of detecting the presence or absence of a genetic variant in a region of interest in the genome of an infant or a fetus in a pregnant woman, wherein the fetal genetic variant is a de novo copy number variant (such as a copy number loss variant) or a paternally-inherited copy number variant (such as a copy number loss variant). In some aspects, the father's genome is sequenced to reveal whether the copy number variant is a paternally inherited copy number variant or a de novo copy number variant. That is, if the fetal copy number variant is not present in the father, and the described method indicates that the fetal copy number variant is distinguishable from the maternal genome, then the fetal copy number variant is a de novo copy number variant. Accordingly, provided herein is a method of determining whether a fetal copy number variant is an inherited copy number variant or a de novo copy number variant.

In some aspects, the methods provided herein allow for detecting the presence or absence of a genetic variant in a region of interest in the genome of an infant or fetus in a pregnant woman, wherein the fetal genetic variant is an autosomal recessive fetal genetic variant. In some aspects, the autosomal fetal genetic variant is an SNP. In some aspects, the fetal genetic variant is a copy number variant, such as a copy number loss variant, or a microdeletion.

In some aspects, the methods provided herein allow for detecting the presence or absence of a genetic variant that is indicative of cancer. A subject having, or suspected of having and/or developing cancer can be assessed and/or treated (e.g., by administering one or more cancer treatments to the subject). In some aspects, a cancer can be an early stage cancer. In some aspects, a cancer can be an asymptomatic cancer. A cancer can be any type of cancer. Examples of types of cancers that can be assessed and/or treated as described herein include, without limitation, lung, colorectal, prostate, breast, pancreas, bile duct, liver, CNS, stomach, esophagus, gastrointestinal stromal tumor (GIST), uterus and ovarian cancer. Additional types of cancers include, without limitation, myeloma, multiple myeloma, B-cell lymphoma, follicular lymphoma, lymphocytic leukemia, leukemia and myelogenous leukemia. In some aspects, the caner is brain or spinal cord tumor, neuroblastoma, Wilms tumor, rhabdomyosarcoma, retinoblastoma or bone cancer, such as osteosarcoma. As such, in some aspects, the cancer is a solid tumor. In some aspects, the cancer is a sarcoma, carcinoma, or lymphoma. In some aspects, the cancer is lung, colorectal, prostate, breast, pancreas, bile duct, liver, CNS, stomach, esophagus, gastrointestinal stromal tumor (GIST), uterus or ovarian cancer. In some aspects, the cancer is a hematologic cancer. In some aspects, the cancer is myeloma, multiple myeloma, B-cell lymphoma, follicular lymphoma, lymphocytic leukemia, leukemia or myelogenous leukemia.

Available treatments for a subject having, or suspected of having, cancer can be administered one or more cancer treatments. A cancer treatment can be any appropriate cancer treatment. One or more cancer treatments described herein can be administered to a subject at any appropriate frequency (e.g., once or multiple times over a period of time ranging from days to weeks). Examples of cancer treatments include, without limitation adjuvant chemotherapy, neoadjuvant chemotherapy, radiation therapy, hormone therapy, cytotoxic therapy, immunotherapy, adoptive T cell therapy (e.g., chimeric antigen receptors and/or T cells having wild-type or modified T cell receptors), targeted therapy such as administration of kinase inhibitors (e.g., kinase inhibitors that target a particular genetic lesion, such as a translocation or mutation), (e.g., a kinase inhibitor, an antibody, a bispecific antibody), signal transduction inhibitors, bispecific antibodies or antibody fragments (e.g., BiTEs), monoclonal antibodies, immune checkpoint inhibitors, surgery (e.g., surgical resection), or any combination of the above. In some aspects, a cancer treatment can reduce the severity of the cancer, reduce a symptom of the cancer, and/or to reduce the number of cancer cells present within the subject.

In some aspects, a subject is treated using an available therapeutic intervention (e.g., treatment), such as, surgery, diet, drug, genetic/gene therapies, device, adjuvant chemotherapy, neoadjuvant chemotherapy, radiation therapy, hormone therapy, cytotoxic therapy, immunotherapy, adoptive T cell therapy and/or targeted therapy.

The term “mutant,” “variant” or “genetic variant,” when made in reference to an allele or sequence, generally refers to an allele or sequence that does not encode the phenotype most common in a particular natural population. In some cases, a mutant allele can refer to an allele present at a lower frequency in a population relative to the wild-type allele. In some cases, a mutant allele or sequence can refer to an allele or sequence mutated from a wild-type sequence to a mutated sequence that presents a phenotype associated with a disease state and/or drug resistant state. Mutant alleles and sequences may be different from wild-type alleles and sequences by only one base but can be different up to several bases or more. The term mutant when made in reference to a gene generally refers to one or more sequence mutations in a gene, including a point mutation, a single nucleotide polymorphism (SNP), an insertion, a deletion, a substitution, a transposition, a translocation, a copy number variation, or another genetic mutation, alteration or sequence variation.

In general, the term “genetic variant” or “sequence variant” refers to any variation in sequence relative to one or more reference sequences. Typically, the variant occurs with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the variant occurs with a low frequency in the population (also referred to as a “rare” sequence variant). For example, the variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some cases, the variant occurs with a frequency of about or less than about 0.1%. A variant can be any variation with respect to a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a variant includes two or more nucleotide differences, the nucleotides that are different may be contiguous with one another, or discontinuous. Non-limiting examples of types of variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of variants include those that occur within short tandem repeats (STR) and simple sequence repeats (SSR), or those occurring due to amplified fragment length polymorphisms (AFLP) or differences in epigenetic marks that can be detected (e.g. methylation differences). In some aspects, a variant can refer to a chromosome rearrangement, including but not limited to a translocation or fusion gene, or fusion of multiple genes resulting from, for example, chromothripsis.

The method of the disclosure contemplates genetic sequencing. Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent™ sequencing), nanopore sequencing, pyrosequencing (454), sequencing by synthesis, sequencing by ligation (SOLID™ sequencing), sequencing by electron microscopy, dideoxy sequencing reactions (Sanger method), massively parallel sequencing, polony sequencing, and DNA nanoball sequencing. In some aspects, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some aspects, the sequencing includes obtaining paired end reads.

In some aspects, sequencing of the nucleic acid from the sample is performed using whole genome sequencing (WGS) or rapid WGS (rWGS®). In some aspects, targeted sequencing is performed and may be either DNA or RNA sequencing. The targeted sequencing may be to a subset of the whole genome. In some aspects, the targeted sequencing is to introns, exons, non-coding sequences or a combination thereof. In other aspects, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain aspects, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. In some aspects, the methodology of the disclosure utilizes systems such as those provided by Illumina, Inc. (HiSeq™ X10, HiSeq™ 1000, HiSeq™ 2000, HiSeq™ 2500, HiSeq™ 4000, NovaSeq™ 6000, Genome Analyzers™, MiSeq™ systems), Applied Biosystems Life Technologies (ABI PRISM™ Sequence detection systems, SOLID™ System, Ion PGM™ Sequencer, ion Proton™ Sequencer).

In some aspects, rWGS® of DNA is performed. In some aspects, rWGS® is performed on samples of the subject, e.g., an infant, neonate or fetus. In some aspects, rWGS® is performed on maternal samples along with that of the subject. In some aspects, rWGS® is performed on paternal samples along with that of the subject. In some aspects, rWGS® is performed on maternal and paternal samples along with that of the subject.

In some aspects, rapid whole exome sequencing (rWES) of DNA is performed. In some aspects, rWES is performed on samples of the subject, e.g., an infant, neonate or fetus. In some aspects, rWES is performed on maternal samples along with that of the subject. In some aspects, rWES is performed on paternal samples along with that of the subject. In some aspects, rWES is performed on maternal and paternal samples along with that of the subject.

As used herein, the term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve whole chromosomes (e.g., aneuploidy). Mutations can involve small sections of DNA. Examples of mutations involving small sections of DNA include, e.g., point mutations or single nucleotide polymorphisms (SNPs), multiple nucleotide polymorphisms, insertions (e.g., insertion of one or more nucleotides at a locus but less than the entire locus), multiple nucleotide changes, deletions (e.g., deletion of one or more nucleotides at a locus), and inversions (e.g., reversal of a sequence of one or more nucleotides). The consequences of a mutation include, but are not limited to, the creation of a new character, property, function, phenotype or trait not found in the protein encoded by the reference sequence. In some aspects, the reference sequence is a parental sequence. In some aspects, the reference sequence is a reference human genome, e.g., h19. In some aspects, the reference sequence is derived from a non-cancer (or non-tumor) sequence. In some aspects, the mutation is inherited. In some aspects, the mutation is spontaneous or de novo.

As used herein, a “gene” refers to a DNA segment that is involved in producing a polypeptide and includes regions preceding and following the coding regions as well as intervening sequences (introns) between individual coding segments (exons).

The terms “polynucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Polynucleotides may be single- or multi-stranded (e.g., single-stranded, double-stranded, and triple-helical) and contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, and the like). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂(“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition including amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, synthetic amino acids and the like), as well as other modifications known in the art.

As used herein, the term “sample” herein refers to any substance containing or presumed to contain nucleic acid. The sample can be a biological sample obtained from a subject. The nucleic acids can be RNA, DNA, e.g., genomic DNA, mitochondrial DNA, viral DNA, synthetic DNA, or cDNA reverse transcribed from RNA. The nucleic acids in a nucleic acid sample generally serve as templates for extension of a hybridized primer. In some aspects, the biological sample is a biological fluid sample. The fluid sample can be whole blood, plasma, serum, ascites, cerebrospinal fluid, sweat, urine, tears, saliva, buccal sample, cavity rinse, feces or organ rinse. The fluid sample can be an essentially cell-free liquid sample (e.g., plasma, serum, sweat, urine, and tears). In other aspects, the biological sample is a solid biological sample, e.g., feces or tissue biopsy, e.g., a tumor biopsy. A sample can also include in vitro cell culture constituents (including but not limited to conditioned medium resulting from the growth of cells in cell culture medium, recombinant cells and cell components). In some aspects, the sample is a biological sample that is a mixture of nucleic acids from multiple sources, i.e., there is more than one contributor to a biological sample, e.g., two or more individuals. In one aspect, the biological sample is a dried blood spot.

In the present invention, the subject is typically a human but also can be any species with methylation marks on its genome, including, but not limited to, a dog, cat, rabbit, cow, bird, rat, horse, pig, or monkey. In one aspect, the subject is a human child. In some aspects, the child is less than 5, 4, 3, 2 or 1 year of age. In aspects, the subject is an infant, neonate or fetus.

### Computer Systems

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in the medical diagnosis context, the present invention may be practiced in conjunction with any number of applications, environments and data analyses; the systems described herein are merely exemplary applications for the invention.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system including a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, include any suitable computer system and associated equipment and may be configured in any suitable manner. In one aspect, the computer system includes a stand-alone system. In another aspect, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing, analysis, reporting and/or diagnosis. The present genetic analysis system maintains information relating to samples and facilitates analysis and/or diagnosis. For example, in the present embodiment, the computer system executes the computer program, which may receive, store, search, analyze, and report information relating to the genome. The computer program may include multiple modules performing various functions or operations, such as a processing module for processing raw data and generating supplemental data and an analysis module for analyzing raw data and supplemental data to generate a disease status model and/or diagnosis information.

The procedures performed by the genetic analysis system may include any suitable processes to facilitate genetic analysis and/or disease diagnosis. In one embodiment, the genetic analysis system is configured to establish a disease status model and/or determine disease status in a patient. Determining or identifying disease status may include generating any useful information regarding the condition of the patient relative to the disease, such as performing a diagnosis, providing information helpful to a diagnosis, assessing the stage or progress of a disease, identifying a condition that may indicate a susceptibility to the disease, identify whether further tests may be recommended, predicting and/or assessing the efficacy of one or more treatment programs, or otherwise assessing the disease status, likelihood of disease, or other health aspect of the patient.

The genetic analysis system may also provide various additional modules and/or individual functions. For example, the genetic analysis system may also include a reporting function, for example to provide information relating to the processing and analysis functions. The genetic analysis system may also provide various administrative and management functions, such as controlling access and performing other administrative functions. The genetic analysis system may also provide clinical decision support, to assist the physician in the provision of individualized genomic or precision medicine for the analyzed patient.

The genetic analysis system suitably generates a disease status model and/or provides a diagnosis for a patient based on genomic data and/or additional subject data relating to the subject's health or well-being. The genetic data may be acquired from any suitable biological samples.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example 1

### Rapid Genome Sequencing for Genetic Disease Diagnosis

In this example, a prototypic, autonomous system for rapid diagnosis of genetic diseases in intensive care unit populations is described. It performs clinical natural language processing (CNLP) to automatically identify deep phenomes of acutely ill children from electronic medical records (EMR).

### Experimental Materials and Methods

### Study Design.

This study was designed to furnish training and test datasets to assist in the development of a prototypic, autonomous system for very rapid, population-scale, provisional diagnoses of genetic diseases by genomic sequencing, and separate datasets to test the analytic and diagnostic performance of the resultant system both retrospectively and prospectively. The 401 subjects analyzed herein were a convenience sample of the first symptomatic children who were enrolled in four studies that examined the diagnostic rate, time to diagnosis, clinical utility of diagnosis, outcomes, and healthcare utilization of rapid genomic sequencing at Rady Children's Hospital, San Diego, USA (ClinicalTrials.gov Identifiers: NCT03211039, NCT02917460, and NCT03385876). One of the studies was a randomized controlled trial of genome and exome sequencing (NCT03211039); the others were cohort studies. All subjects had a symptomatic illness of unknown etiology in which a genetic disorder was suspected. All subjects had a Rady Children's Hospital Epic EHR and a genomic sequence (genome or exome) that had been interpreted manually for diagnosis of a genetic disease. They included five groups, namely, 16 children tested for genetic diseases by rapid whole genome sequencing whose EHRs were used to train CNLP (Table 4), ten children with genetic diseases diagnosed by rapid genomic sequencing whose EHRs were used to test the performance of CNLP (Table 5), 101 children with genetic diseases diagnosed by rapid genomic sequencing whose genomic sequences and EHRs were used to test the retrospective performance of the autonomous diagnostic system, seven seriously ill children with suspected genetic diseases whose DNA samples and EHRs were used to test the prospective performance of the autonomous diagnostic system (Table 1), and 274 control children in whom rapid genomic sequencing did not disclose a genetic disease diagnosis.

### Standard, Clinical, Rapid Whole Genome and Exome Sequencing, Analysis and Interpretation.

Standard, clinical, rWGS® and rWES were performed in laboratories accredited by the College of American Pathologists (CAP) and certified through Clinical Laboratory Improvement Amendments (CLIA). Experts selected key clinical features representative of each child's illness from the Epic EHR and mapped them to genetic diagnoses with Phenomizer™ or Phenolyzer™. Trio EDTA-blood samples were obtained where possible. Genomic DNA was isolated with an EZ1 Advanced XL™ robot and the EZ1 DSP DNA™ Blood kit (Qiagen). DNA quality was assessed with the Quant-iT Picogreen dsDNA™ assay kit (ThermoFisher Scientific) using the Gemini EM Microplate Reader™ (Molecular Devices). Genomic DNA was fragmented by sonication (Covaris) and bar-coded, paired-end, PCR-free libraries were prepared for rWGS® with TruSeq DNA LT™ kits (Illumina) or Hyper kits (KAPA Biosystems). Sequencing libraries were analyzed with a Library Quantification Kit™ (KAPA Biosystems) and High Sensitivity NGS Fragment Analysis Kit™ (Advanced Analytical), respectively. Paired-end 101 nt rWGS® was performed to 45-fold coverage with Illumina HiSeq™ 2500 (rapid run mode), HiSeq™ 4000, or NovaSeq™ 6000 (S2 flow cell) instruments, as described. rWES was performed by GeneDx™. Exome enrichment was with the xGen Exome Research Panel™ v1.0 (Integrated DNA Technologies), and amplification used the Herculase II Fusion™ polymerase (Agilent). Sequences were aligned to human genome assembly GRCh37 (hg19), and variants were identified with the DRAGEN™ Platform (v.2.5.1, Illumina, San Diego). Structural variants were identified with Manta™ and CNVnator™ (using DNAnexus™), a combination that provided the highest sensitivity and precision in 21 samples with known structural variants (Table 6). Structural variants were filtered to retain those affecting coding regions of known disease genes and with allele frequencies <2% in the RCIGM database. Nucleotide and structural variants were annotated, analyzed, and interpreted by clinical molecular geneticists using Opal Clinical™ (Fabric Genomics), according to standard guidelines. Opal™ annotated variants with respect to pathogenicity, generated a rank ordered differential diagnosis based on the disease gene algorithm VAAST, a gene burden test, and the algorithm PHEVOR (Phenotype Driven Variant Ontological Re-ranking), which combined the observed HPO phenotype terms from patients, and re-ranked disease genes based on the phenotypic match and the gene score. Automatically generated, ranked results were manual interpreted through iterative Opal searches. Initially, variants were filtered to retain those with allele frequencies of <1% in the Exome Variant Server™, 1000 Genomes Samples™, and Exome Aggregation Consortium™ database. Variants were further filtered for de novo, recessive and dominant inheritance patterns. The evidence supporting a diagnosis was then manually evaluated by comparison with the published literature. Analysis, interpretation and reporting required an average of six hours of expert effort. If rWGS® or rWES established a provisional diagnosis for which a specific treatment was available to prevent morbidity or mortality, this was immediately conveyed to the clinical team, as described. All causative variants were confirmed by Sanger sequencing or chromosomal microarray, as appropriate. Secondary findings were not reported, but medically actionable incidental findings were reported if families consented to receiving this information.

### Natural Language Processing and Phenotype Extraction.

Extraction of HPO terms from the EHR entailed four steps as follows.

1) Clinical records were exported from the EHR data warehouse, transformed into a compatible format (JSON) and loaded into CLIX ENRICH™.

2) A semi-automated query map was created, using HPO terms (and their synonyms) as the input and CLIX queries as the output. The HPO terms were passed through the CLIX encoding engine, resulting in creation of CLIX post-coordinated SNOMED™ expressions for each recognized HPO term or synonym. Where matches were not exact, manual review was used to validate the generated CLIX™ queries. Where there was no match or incorrect matches, new content was added to the Clinithink SNOMED™ extension and terminology files to ensure appropriate matches between phenotypes in HPO and those in SNOMED-CT™. This was an iterative process that resulted in a CLIX™ query set that covered 60% (7,706) of 12,786 HPO terms (Oct. 9 2017 HPO build).

3) EHR documents containing unstructured data were passed through the CNLP engine. The natural language processing engine read the unstructured text and encoded it in structured format as post-coordinated SNOMED expressions as shown in the example below which corresponds to HP0007973, retinal dysplasia:
243796009|Situation with explicit context: {408731000|Temporal context|=410511007|Current or past|, 246090004 Associated finding|=95494009|Retinal dysplasia, 408732007|Subject relationship context|=410604004|Subject of record, 408729009| Finding context|=410515003 Known present|}

Each SNOMED expression is made up of several parts, including the associated clinical finding, the temporal context, finding context and subject context all contained within the situational wrapper. Capturing fully post-coordinated SNOMED expressions ensures that the correct context of the clinical note is preserved. Some HPO phenotypes cannot be found in SNOMED and can only be represented using post-coordinated expressions, as shown in the following example, which is the encoding of HP0008020, progressive cone dystrophy:
243796009|Situation with explicit context|: {408731000|Temporal context|=410511007|Current or past, 246090004|Associated finding|=(312917007|Cone dystrophy|: 263502005|Clinical course|=255314001|Progressive|), 408732007|Subject relationship context|=410604004|Subject of record|, 408729009|Finding context|=410515003|Known present|}

Here, an additional attribute for ‘Clinical Course’ and an appropriate value, ‘Progressive’, are used to further qualify the expression. Clinithink™ used references to these SNOMED™ expressions, linked with Boolean logic, to create the queries corresponding to HPO terms. Shown below is an example query for HP0008866, failure to thrive secondary to recurrent infections:
c*hp0008866_Failure_to_thrive_secondary_to_recurrent_infections (hp0008866_1_1_Failure_to_thrive_q AND hp0002719_1_1_Infection_Recurrent_q)
q-hp0008866_1_1_Failure_to_thrive_q 243796009 Situation with explicit context|:{408731000|Temporal context|=410511007|Current or past|,246090004|Associated finding|=54840006|Failure to thrive|,408732007|Subject relationship context|=410604004|S ubject of record|,408729009|Finding context|=410515003|Known present|}
q-hp0002719_1_1_Infection_Recurrent_q 243796009|Situation with explicit context|:{408731000|Temporal context|=410511007|Current or past|,246090004|Associated finding|=(40733004|Infection|:263502005|Clinical course|=255227004|Recurrent|), 4087320 07|Subject relationship context|=410604004|Subject of record|, 408729009|Finding context|=410515003|Known present|}

For an encoding created from the unstructured data to trigger one of these queries, all of the components must be matched. Therefore, the encoding of a clinical note describing an affected sibling will not trigger the query since the encoding is that of family history whilst the query looks for the term in the subject of the record (e.g., the patient). Furthermore, it should be noted that some individual HPO synonyms generate more than one SNOMED™ expression. Therefore, each query used in the query set is a compound of often more than 2 SNOMED™ expressions. If the above constants are stripped out from each expression (the associated clinical finding, the temporal context, finding context and subject context all contained within the situational wrapper) from each expression in the query set (along with all of the associated SNOMED™ codes), the inventors can create a more readable format to show linguistically what is included in each query created by Clinithink™.

4) This encoded data was then interrogated by the CLIX™ query technology (abstraction). To trigger an HPO query, the encoded data had to either contain an exact match, or one of its logical descendants (exploiting the parent child hierarchy of the SNOMED™ ontology), resulting in a list of HPO terms for each patient.

rWGS®.

Sequencing libraries were prepared from 10 μL of EDTA blood or five 3-mm punches from a Nucleic-Card Matrix™ dried blood spot (ThermoFisher) with Nextera DNA Flex Library Prep™ kits (Illumina) and five cycles of PCR, as described. For structural variant analysis, libraries were prepared by Hyper™ kits (KAPA Biosystems), as described above. Libraries were quantified with Quant-iT Picogreen dsDNA™ assays (ThermoFisher). Libraries were sequenced (2×101 nt) without indexing on the S1 FC with Novaseq™ 6000 S1 reagent kits (Illumina). Sequences were aligned to human genome assembly GRCh37 (hg19), and nucleotide variants were identified with the DRAGEN™ Platform (v.2.5.1, Illumina).

### Automated Tertiary Analysis.

Automated variant interpretation was performed using MOON™ (Diploid). Data sources and versions were ClinVar™: 2018 Apr. 29; dbNSFP: 3.5; dbSNP: 150; dbscSNV: 1.1; Apollo: 2018 Jul. 20; Ensembl: 37; gnomAD: 2.0.1; HPO: 2017 Oct. 5; DGV: 2016 Mar. 1; dbVar: 2018 Jun. 24; MOON: 2.0.5). MOON™ generated a list of potential provisional diagnoses by sequentially filtering and ranking variants using decision trees, Bayesian models, neural networks, and natural language processing. MOON™ was iteratively trained with thousands of prior patient samples uploaded by prior investigators. No samples analysed in this study were used in training of MOON™.

The filtering pipeline was designed to minimize false negatives. For SNV analysis, MOON™ excluded low quality and common variants (>2% in gnomAD), and known likely benign/Benign variants in ClinVar™. Only variants in coding regions, splice site regions and known pathogenic variants in non-coding regions were retained. A disease annotation was added to the remaining variants based on a proprietary disorder model. The disorder model performs natural language processing of the genetics literature to automatically extract associations between diseases, disease genes, inheritance patterns, specific clinical features, and other metadata on an ongoing basis.

Subsequent steps included filtering on variant frequency, with variable frequency thresholds depending on the inheritance pattern of the associated disease, known pathogenicity of the variant, and typical age of onset range of the annotated disease. In family analyses (duo/trio analysis), co-segregation of the variant with the phenotype, according to autosomal dominant, autosomal recessive, X-linked dominant or X-linked recessive inheritance patterns, was taken into account. Parent-child variant segregation was not applied as a strict filter criterion, thereby also ensuring that causal mutations following non-Mendelian inheritance (eg. with incomplete penetrance) were identified in family analyses. For proband-only analyses, only variants for which the zygosity of the called variant fit the inheritance pattern of the annotated disease were retained. In a final filter step, the phenotype overlap was scored between the input HPO terms describing the patient's phenotype and known disease manifestations of the annotated disorder annotated from the published literature. Variants in genes for which the phenotype match with the annotated disease was considered too limited based on Apollo™ were removed from the analysis. The final rank of variants was based on proprietary algorithms that took phenotype match and variant effect into account. In addition, MOON™ provided all metadata supporting the pathogenicity of ranked variants. MOON™ also returned an annotated list of all rare variants (<2% in gnomAD) and carrier status for recessive disorders.

For structural variant analysis, MOON™ removed known benign SV based on the Database of Genomic Variants™ (DGV). SVs overlapping pathogenic SVs listed in dbVar were retained for analysis. From the remaining variants, MOON™ discarded SV that did not overlap with coding regions of known disease genes (Apollo™). If a family analysis was performed, segregation of the SV was taken into account, although non-Mendelian inheritance patterns (for example, incomplete penetrance) were also supported. In a final filter step, only SVs for which there was phenotype overlap between the input HPO terms and known disease presentations of at least one of the genes affected by the SV, were retained. MOON™ then reported a ranked list of candidate SV, where ranking was mostly based on phenotype overlap.

### Statistical Analysis.

To assess the complexity of phenomes associated with childhood genetic diseases, the inventors compared phenotypes identified by manual review, CNLP, and listed for each patient's diagnosis in OMIM. All analyses were conducted in R v3.3.3. When applying CNLP to a patient's EHR, the list of HPO terms produced contained both terms that had an exact match to a phenotype in the clinical notes and terms that were superclasses (ancestor terms) of exact matches. The R package ontologyIndex™ v2.4 was used to load the October 2017 build of HPO into R and calculate the IC of each HPO term in the entire OMIM corpus. The IC for term phenotype, which reflects its clinical specificity, is given by IC (phenotype)=−log (p_{phenotype}), where p_{phenotype} was the probability of observing the exact term or one of its subclasses across all diseases in OMIM™. Since phenotypes that were extracted manually and by CNLP were restricted to subclasses of ‘Phenotypic abnormality’ (HP:0000118), OMIM™ terms that were subclasses of ‘Clinical Modifier’ (HP:0012823), ‘Frequency’ (HP:0040279), ‘Mode of inheritance’ (HP:0000005), and ‘Mortality/Aging’ (HP:0040006) were not included in the analyses. Phenotype sets were first compared visually by plotting the HPO graph for each patient with the R package hpoPlot™ v2.4. Summary statistics for outcomes of interest include the mean, standard deviation (SD), and range. Prior to testing for significant differences, outcome variables were tested for normality using the Shapiro-Wilk test. Due to deviations from normality, differences in phenotype counts and IC were evaluated with 2-sided Mann-Whitney U tests and when the data were paired, Wilcoxon signed-rank tests. Correlation was assessed with Spearman's rank correlation coefficient (rₛ). Precision and recall were given by tp/(tp+fp) and tp/(tp+fn), respectively, where tp were true positives, fp were false positives, and fn were false negatives. The number of true positives, tp, was defined in two ways. First, tp was set to the number of HPO terms that overlapped between sets of phenotypes. Second, tp was calculated based on terms that were up to one degree of separation apart within the HPO hierarchy (parent-child terms) between sets of phenotypes, allowing for inexact, but similar, matches. Additional graphics were produced with packages ggplot2 v 2.2.1 and eulerr v4.0.0. A significance cutoff of p<0.05 was used for all analyses.

### Results

### Rapid Genome Sequencing for Genetic Disease Diagnosis.

In light of the limitations of current methods of rapid genomic sequencing, the inventors developed an automated platform for rapid, high throughput, provisional diagnosis of genetic diseases with genome sequencing by automating and accelerating our conventional workflow (FIG. 1). Conventional clinical genome sequencing requires preparatory steps of manual purification of genomic DNA from blood, DNA quality assessment, normalization of DNA concentration, sequencing library preparation, and library quality assessment (FIG. 1A). Instead, the inventors manually prepared sequencing libraries directly from blood or dried blood spots using microbeads to which transposons were attached (Nextera DNA Flex Library Prep Kit™, Illumina, Inc.; FIG. 1B), as this method was both faster and less labor intensive. Of note, dried blood spots are the sample type used in mandatory newborn screening worldwide. In four timed runs with retrospective samples, manual Nextera™ library preparation from dried blood spots took a mean of 2 hours and 45 minutes, compared with at least 10 hours by conventional DNA purification and library preparation (Truseq DNA PCR-free Library Prep Kit™, Illumina, Inc.; Table 1). As with standard methods, Nextera Flex™ allowed samples to be prepared in batches and was amenable to automation with liquid-handling robots.

Following the preparatory steps, our previous method performed rapid genome sequencing with the HiSeq™ 2500 sequencer (Illumina) in rapid run mode, with one sample sequenced per sequencing instrument (˜120 gigabases (Gb) of 2×101 nt) in ˜25 hours (FIG. 1A). Here the inventors instead performed rapid genome sequencing with the NovaSeq™ 6000 sequencer and S1 flow cell (Illumina) (FIG. 1B), as this instrument was faster and less labor-intensive, requiring fewer steps to set up a sequencing run and automatically washing the instrument after a run. In four timed runs with retrospective samples, 2×101 nt genome sequencing took a mean 15:32 hours and yielded 404-537 Gb per flow cell, sufficient for 2-3 40× genome sequences (Table 1, Table 2).

Dynamic Read Analysis for GENomics™ (DRAGEN™, Illumina) is a hardware and software platform for alignment and variant calling that has been highly optimized for speed, sensitivity and accuracy. The inventors wrote scripts to automate the transfer of files from the sequencer to the DRAGEN™ platform. The DRAGEN™ platform then automatically aligned the reads to the reference genome and identified and genotyped nucleotide variants. Alignment and variant calling took a median of 1 hour for 150 Gb of paired-end 101 nt sequences (primary and secondary analysis, Table 1). Analytic performance of this new method, from blood sample receipt to output of genomic variant genotypes, was similar to standard clinical methods with reference human genome samples, retrospective patient samples, and prospective patient samples, except for lower sensitivity in the detection of nucleotide insertions/deletions (Table 2, Table 3). The new method did not assess structural variations.

### CNLP of Electronic Health Records (EHRs).

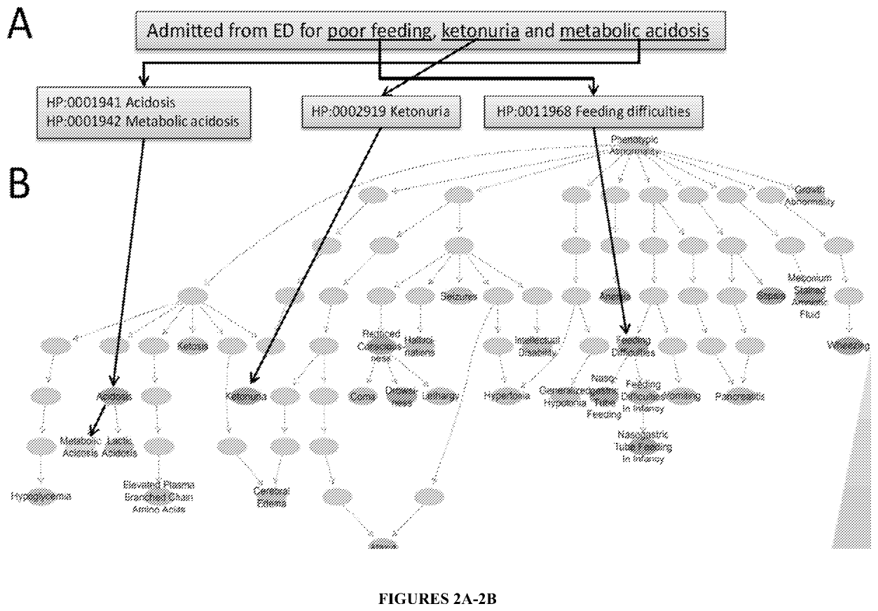

Genetic disease diagnosis requires determination of a differential diagnosis based on the overlap of the observed clinical features of a child's illness (phenotypic features) with the expected features of all genetic diseases. However, comprehensive EHR review can take hours. Additionally, manual phenotypic feature selection can be sparse and subjective, and even expert reviewers can carry an unwritten bias into interpretation (FIG. 1A). The inventors sought automated, complete phenotypic feature extraction from EHRs, unbiased by expert opinion. The simplest approach would be to extract universal, structured phenotypic features, such as International Classification of Diseases (ICD) medical diagnosis codes, or Diagnosis Related Group (DRG) codes. However, these are sparse and lack sufficient specificity. Instead, the inventors extracted clinical features from unstructured text in patient EHRs by CNLP that the inventors optimized for identification of patients with orphan diseases (CLIX ENRICH™, Clinithink Ltd.) (FIG. 1B, 2A). The inventors then iteratively optimized the protocol for the Rady Children's Hospital Epic EHRs using a training set of sixteen children who had received genomic sequencing for genetic disease diagnosis (Table 4). The standard output from CLIX ENRICH™ is in the form of Systematized Nomenclature of Medicine Clinical Terms (SNOMED-CT™). However, our automated methods required phenotypic features described in the Human Phenotype Ontology (HPO), a hierarchical reference vocabulary designed for description of the clinical features of genetic diseases (FIG. 2B). For this reason, the inventors mapped 7,706 (60%) of 12,786 HPO terms (13,685 including synonyms) and 75.4% of Orphanet Rare Disease HPO™ terms (June 2018 release) to SNOMED-CT™ by lexical and logical methods and then manually verified them. This enabled automated translation of phenotypic features extracted from the EHR by CNLP from SNOMED-CT™ concepts to HPO™ terms (FIG. 1B). In contrast, a previous study mapped 92% of HPO™ terms to SNOMED-CT™, but only 49% were shown to be ontologically valid and clinically relevant.

The performance of the optimized CNLP was tested with the EHRs of ten test children who had received genomic sequencing for genetic disease diagnosis. The training and test sets did not overlap. Both exact EHR phenotypic feature matches and their hierarchical root terms were extracted from first record until time of enrollment for genomic sequencing. CNLP identified a mean of 86.7 phenotypic features (standard deviation (SD) 32.8, range 26-158; Table 5) in approximately 20 seconds per patient. A detailed manual review of the EHR was performed to identify all true positive, false positive and false negative CNLP phenotypic features in the test children. Based on this, the precision (positive predictive value, PPV) of CNLP was 0.80 (SD 0.13, range 0.50-0.93) and recall (sensitivity) was 0.93 (SD 0.02, range 0.91-0.96; Table 5), which were superior to prior CNLP-based extraction of HPO terms. The principal reasons for false positives (FP) were: 1) incorrect CLIX™ encoding (n=89, 38% of 237 phenotypic features) due to misinterpreted context (n=31), unrecognized headings (n=23), incorrect acronym expansion (n=21), incorrect interpretation of a clinical word (n=8), or incorrectly attributed finding site for disease (n=6); 2) ambiguity of source text (unrecognized or incorrect syntax, abbreviations, acronyms or terminology; n=46, 19% of 237); 3) incongruity between SNOMED/HPO/clinical acumen (n=20, 8%); 4) failure to recognize a pasted citation as non-clinical text (n=68, 29%); and, 5) incorrect query logic (n=14, 6%) (Table 5).

Characterization of the CNLP-Derived Phenomes of Children with Suspected Genetic Diseases.

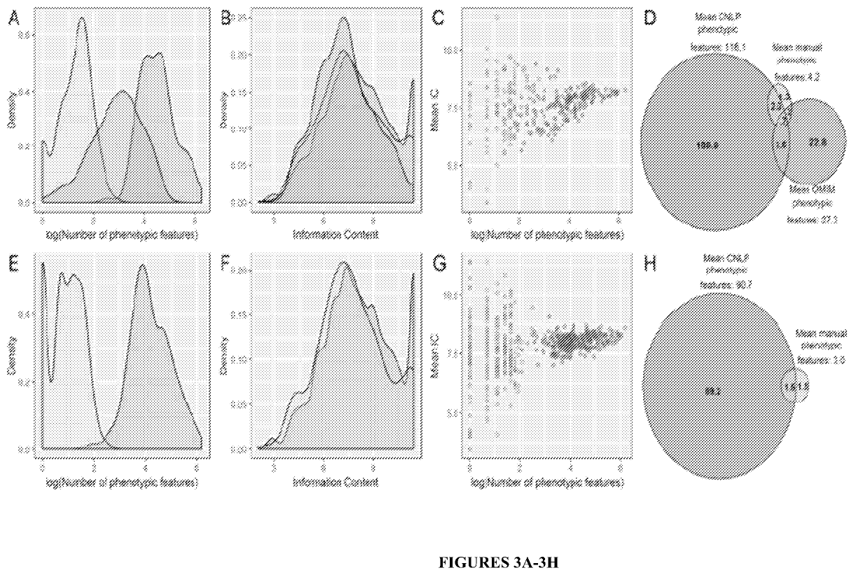

Development of an autonomous diagnostic system has been hindered by a dearth of knowledge of the topography of the phenomes of children with suspected genetic diseases. Therefore the inventors compared EHR CNLP-derived phenomes with the comparatively sparse phenotypic features selected by experts during manual interpretation of the first 375 symptomatic children to receive genomic sequencing for diagnosis of genetic diseases at Rady Children's Hospital (101 children diagnosed with genomic sequencing: FIGS. 3A-D, 274 children that were not diagnosed: FIG. 3E-H). In 101 of these children, who had received genomic diagnoses of 105 genetic diseases (four had dual diagnoses), the inventors also compared the observed phenotypic features with the expected phenotypic features for those diseases, obtained from the Clinical Synopsis field of Online Mendelian Inheritance in Man™ (OMIM™). In the 101 diagnosed children, CNLP identified 27-fold more phenotypic features (mean 116.1, SD 93.6, range 13-521) than expert manual selection at interpretation (mean 4.2, SD 2.6, range 1-16), and 4-fold more than OMIM (mean 27.3, SD 22.8, range 1-100; FIG. 3A, 3D) (45, 46). Similarly, prior studies demonstrated 2-fold more phenotypic features extracted by CNLP than comprehensive, expert manual extraction, and 18-fold more phenotypic features extracted by CNLP than Orphanet HPO™ terms for those diseases. CNLP extracted more phenotypic features in the 101 diagnosed children than the 274 undiagnosed children (mean, 116.1 vs 90.7, respectively; P=0.0004, Mann-Whitney U test; FIG. 3A, 3D, 3E, 3H). This suggested the possibility that undiagnosed children, in part, did not have enough detail in their medical records to make a molecular diagnosis. In addition, there was greater overlap between CNLP- and manually-extracted phenotypic features in diagnosed children (mean 2.74 terms, SD 1.7, range 0-9) than undiagnosed (mean 1.52 terms, SD 1.48, range 0-7; P<0.0001, Mann-Whitney U test; FIG. 3D, 3H). This suggested that undiagnosed children, in part, had less consistent information on phenotypic features.

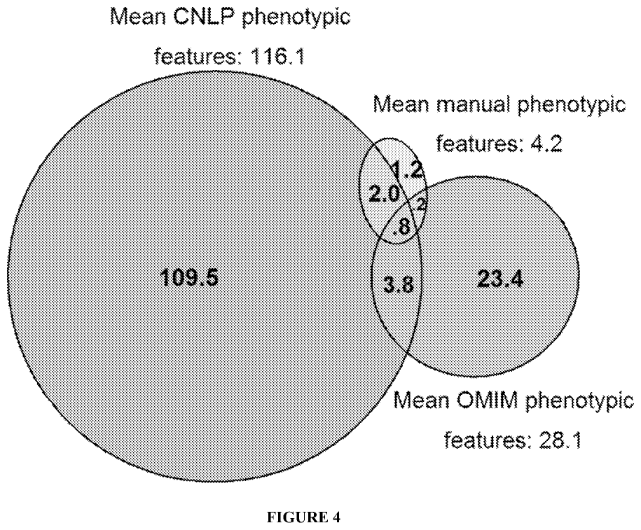

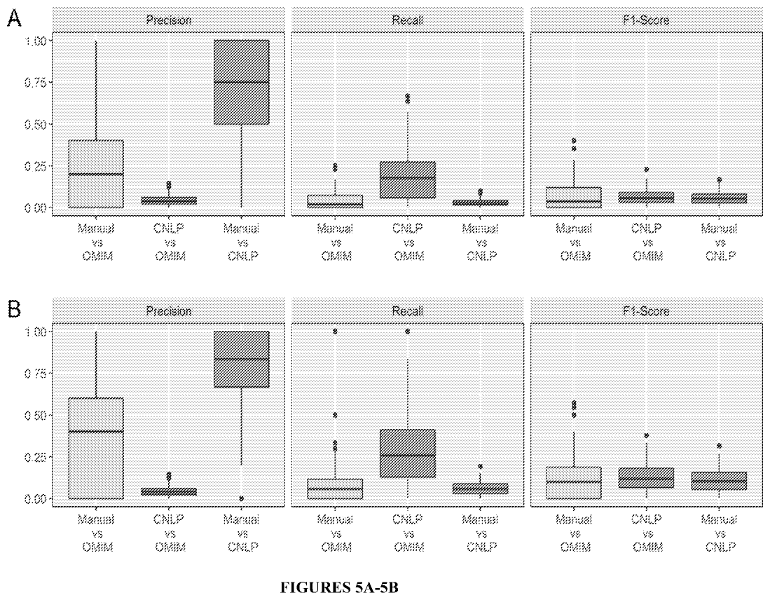

In the 101 diagnosed children, phenotypic features extracted by CNLP overlapped expected OMIM phenotypic features (mean 4.31 terms, SD 4.59, range 0-32) significantly more than the manual extracted phenotypic features (mean 0.92 terms, SD 1.02, range 0-4; P<0.0001, paired Wilcoxon test; FIG. 3B). Although the cohort included eight genetic diseases that were incidental findings, their exclusion did not materially change these results (FIG. 4). Thus, the recall of OMIM™ phenotypic features by CNLP, although small (mean 0.20, SD 0.16, range 0-0.67), was substantially greater than the sparse expert manual phenotypic features used in expert manual interpretation (mean 0.04, SD 0.06, range 0-0.25) (FIG. 5). However, the much larger number of phenotypic features extracted by CNLP was associated with lower precision (mean 0.04, SD 0.03, range 0-0.15) than manual extraction (mean 0.25, SD 0.30, range 0-1) when compared with OMIM, indicating that, by design, an autonomous diagnostic system should not penalize false positive phenotypic features. Recall and F₁ value increased when phenotypic features with one degree of hierarchical separation to those extracted were included (mean CNLP recall with inexact matches 0.29, SD 0.22, range 0-1; mean CNLP F1 with inexact matches 0.12, SD 0.08, range 0-0.38; mean CNLP F1 with exact matches 0.06, SD 0.05, range 0-0.23), indicating that, by design, an autonomous system should include hierarchical parents of extracted terms (FIG. 5).

Traditionally, genetic diseases have been clinically diagnosed by the identification of one or more pathognomonic phenotypic features. Such phenotypic features have high information content (IC, the logarithm of the probability of that phenotypic feature being observed in all OMIM™ diseases; FIG. 2). A potential concern was that phenotypic features extracted by CNLP would have less information content than those prioritized manually by experts during interpretation. However, among the 101 children, the mean IC of CNLP phenotypic features (8.1, SD 2.0, range 2.6-11.4) was significantly higher than manual (7.8, SD 2.0, range 2.1-11.4; P=0.003, Mann-Whitney U test) or OMIM™ phenotypic features (7.3, SD 1.7, range 3.2-11.4; P<0.0001, Mann-Whitney U test, FIG. 3E). The inventors note that the mean IC correlated significantly with number of phenotypic features extracted manually and by CNLP (Spearman's rho 0.24, P=0.02 and Spearman's rho 0.44, P<0.0001, respectively; FIG. 3C). The mean IC of CNLP phenotypic features was higher than manual phenotypic features (FIG. 3F), and the mean IC correlated significantly with number of phenotypic features extracted by CNLP (Spearman's rho 0.30, P<0.0001; FIG. 3G).

### Retrospective Performance of an Autonomous System for Diagnosis of Childhood Genetic Diseases.

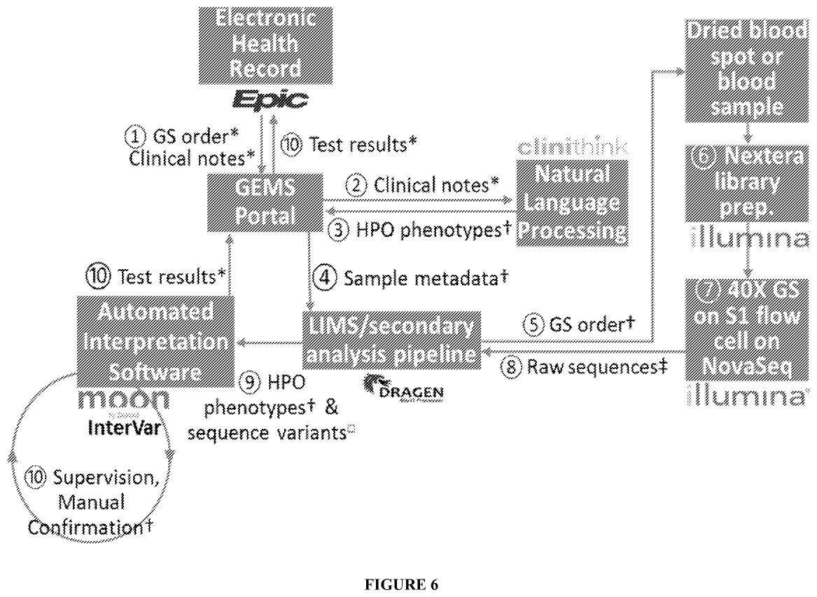

The remaining steps in automated diagnosis of genetic diseases were to combine the automated ranking of the patient's CNLP phenome with respect to all genetic diseases, together with the automated ranking of the pathogenicity of all their genomic variants based on literature knowledge and in silico tools (FIG. 1, FIG. 6). The inventors wrote scripts to transfer the patient's CNLP-derived phenotypic features and genomic variants automatically to autonomous interpretation software (MOON™, Diploid). MOON™ identified the phenotypic features associated with each genetic disease by natural language processing of the medical literature. Typically, this was a larger set of phenotypic features than those listed in the OMIM™ Clinical Synopsis. MOON™ then compared the patient's phenotypic features with those associated with each genetic disease and rank-ordered their likelihood of causing the child's illness.

The inventors also wrote scripts to transfer a patient's nucleotide and structural variants automatically from the DRAGEN™ platform to MOON as soon as it finished, without user intervention. For rapid genome sequencing, there was a mean of 4,742,595 nucleotide variants and 19.3 structural variants (SVs) and exome sequencing had a mean of 39,066 nucleotide variants and 10.3 SVs per patient. Of these, MOON™ retained 67,589 nucleotide variants and 12 SVs, and 791 nucleotide variants and 4.5 SVs, for rapid genome and exome sequencing, respectively, that had allele frequencies <2% and affected known disease genes. A Bayesian framework and probabilistic model in MOON™ ranked the pathogenicity of these variants with 15 in silico prediction tools, ClinVar™ assertions, and inheritance pattern-based allele frequencies. In singleton and family trio analyses, a mean of five and three provisional diagnoses were ranked, respectively (Table 6). Since MOON™ was optimized for sensitivity, it shortlisted a median of 6 nucleotide variants per diagnosed subject (range 2-24), and often shortlisted false positive diagnoses in cases considered negative by manual interpretation. Both were largely remedied, however, by processing the MOON™ output in InterVar™ software, and retaining only pathogenic and likely pathogenic variants. InterVar™ classified variants with regard to 18 of the 28 consensus pathogenicity recommendations, specifically triaging variants of uncertain significance (VUS). Automated interpretation took a median of five minutes from transfer of variants and HPO terms to display of the provisional diagnosis and supporting evidence, including patient phenotypic features matching that disorder, for laboratory director review. In four timed runs, the time from blood or blood spot receipt to display of the correct diagnosis as the top ranked variant was 19:14-20:25 hours (median 19:38 hours, Table 1, retrospective cases). This conformed well to a daily clinical operation cycle: sample receipt in the morning enabled library preparation in the afternoon, genome sequencing overnight, and provisional reporting early the following morning for laboratory director review.

The inventors retrospectively examined the concordance between the autonomous system and prior, team-based, manual expert interpretation in 95 of the 101 children, diagnosed with 97 of the 105 genetic diseases. The inventors excluded 8 findings that had been reported but that were considered incidental (without current evidence of any of the expected phenotypic features). This cohort was diverse in race and ancestry. Eleven diagnoses were associated with structural variants, and 86 with nucleotide variants. No training patients were included in the test set. In two patients, a revised clinical report was issued of a new diagnosis (infant 6007, EIEE9, Xp22 del, and patient 6033, Cockayne syndrome B, ERCC6 p.Gly528Glu and c.−15+3G>T, which was validated by functional studies). Therefore, initial expert manual interpretation had a recall of 98% (95 of 97). Although the inventors did not re-analyze manual diagnoses, none of them had been demoted in the period since initially reported clinically. The autonomous diagnostic system had precision of 99% (93 of 94) and recall of 97% (94 of 97). For nucleotide and structural variants, the median rank of the correct diagnosis was first (range 1-4 nucleotide variants; range 1-13 SV; Table 6).

The three false negative autonomous diagnoses included the following cases.

Infant 6159, with autosomal dominant Alport syndrome (COL4A4 c.4715C>T, p.Pro1572Leu), had hematuria, nephrotic syndrome, glomerulonephritis, hypertension, and anasarca. OMIM™ indicated COL4A4-associated Alport syndrome (CAS) was autosomal recessive, and p.Pro1572Leu was recorded as pathogenic in ClinVar™ for autosomal recessive Alport syndrome. There are, however, a large number of reports of autosomal dominant CAS. The variant was maternally inherited. Since the infant's mother was asymptomatic, the inventors assumed that she exhibited incomplete penetrance of autosomal dominant CAS, as has been reported. The autonomous system classified the infant as a carrier for autosomal recessive CAS.

Infant 253 had autosomal dominant optic atrophy plus syndrome (OPA1 c.556+1G>A). The autonomous system did not rank this variant because of insufficient overlap of the 70 CNLP phenotypic features with the MOON™ disease phenotypic feature model. Recent reports indicate that OPA1 can be associated with complex, severe multi-system mitochondrial disorders, similar to infant 253.

Neonate 213 had dextrocardia and transposition of the great vessels. He received singleton genome sequencing, and was diagnosed manually with autosomal dominant visceral heterotaxy type 5 associated with a likely pathogenic variant in NODAL (c.778G>A; p.Gly260Arg). This variant was filtered out by the autonomous system based on classification as a VUS by InterVar™ (based on PM1-PP3-PP5) and the presence of conflicting interpretations in ClinVar™, including a ‘Likely Benign’ assertion.

When the relatively sparse phenotypic features selected by experts during manual interpretation were substituted for phenotypic features identified by CNLP, the recall of the autonomous system decreased (88%, 85 of 97).

### Prospective Performance of an Autonomous System for Diagnosis of Childhood Genetic Diseases.

The inventors prospectively compared the performance of the autonomous diagnostic system with the fastest manual methods in seven seriously ill infants in intensive care units and three previously diagnosed infants (Table 1). The median time from blood sample to diagnosis with the autonomous platform was 19:56 hours (range 19:10-31:02 hours), compared with the median manual time of 48:23 hours (range 34:38-56:03 hours). This included two automated runs which were delayed by operator error or data center downtime. The autonomous system coupled with InterVar™ post-processing made three diagnoses and no false positive diagnoses. All three diagnoses were confirmed by manual methods and Sanger sequencing. The first was for patient 352, a seven-week-old female, admitted to the pediatric intensive care unit with diabetic ketoacidosis. Rapid genome sequencing was performed on the singleton proband. In 19:11 hours, the autonomous system identified a previously unreported, heterozygous missense variant in the insulin gene (INS c.26C>G, pPro9Arg), which is associated with autosomal dominant permanent neonatal diabetes mellitus (OMIM™ disease record 606176). According to ACMG/AMP pathogenicity criteria, the variant was of uncertain significance (VUS). After 42:04 hours, parent-child trio sequencing with the fastest manual methods confirmed the result and showed the variant to be de novo, which changed the variant classification to likely pathogenic.

The second diagnosis was made in patient 7052, a previously healthy 17-month-old boy admitted to the pediatric intensive care unit with pseudomonal septic shock, metabolic acidosis, echthyma gangrenosum and hypogammaglobulinemia. Singleton, proband, rapid sequencing and automated interpretation identified a pathogenic hemizygous variant in the Bruton tyrosine kinase gene (BTK c.974+2T>C) associated with X-linked agammaglobulinemia 1 (OMIM™: 300755) in 22:04 hours. This was 16:33 hours earlier than a concurrent trio run with the fastest manual methods. The provisional result provided confidence in treatment with high-dose intravenous immunoglobulin (to maintain serum IgG >600 mg/dL) and six weeks of antibiotic treatment. This provisional diagnosis was verbally conveyed to the clinical team upon review of the autonomous result by a laboratory director. Clinical whole genome sequencing subsequently returned the same result and showed the variant to be maternally inherited.

The third diagnosis was made in patient 412, a 3-day-old boy admitted to the neonatal ICU with seizures and a strong family history of infantile seizures responsive to phenobarbital. The autonomous system identified a likely pathogenic, heterozygous variant in the potassium voltage-gated channel, KQT-like subfamily, member 2 gene (KCNQ2 c.1051C>G). This gene is associated with autosomal dominant benign familial neonatal seizures 1 (OMIM™ disease record 121200). The diagnosis was made in 20:53 hours, which was 27:30 hours earlier than a concurrent run with the fastest manual methods. A verbal provisional result was conveyed to the clinical team upon review of the result by a laboratory director as the diagnosis provided confidence in treatment with phenobarbital and changed the prognosis.

For the remaining four patients, no diagnosis was evident with either manual or autonomous methods.

### Discussion

Previously, the fastest time to diagnosis by genome sequencing in clinical practice was 37 hours. The protocol was, however, extremely labor- and capital-intensive, and limited to one sample at a time. Here the inventors described a prototypic, autonomous system for genetic disease diagnosis in a median of 20:10 hours requiring decreased user intervention and a throughput of up to two parent-child trios or six probands per run. Most decision making in ICUs is made deliberatively in morning rounds attended by a multidisciplinary healthcare team. Thus, a 20-hour diagnosis would return results to the on-call physician who ordered testing in time for morning rounds. This would simplify information transfer during rounds and facilitate management decisions. A 20-hour diagnosis is important in seriously ill infants as a majority of timely genomic diagnoses result in changes in ICU management.

The autonomous platform for 20-hour diagnosis of genetic diseases was designed to meet the needs of acutely ill infants in ICUs with diseases of unknown etiology. It has been estimated that 10-12% of infants admitted to regional ICUs may benefit from same-day diagnosis and implementation of targeted treatments. In 2014, the US Food and Drug Administration (FDA) permitted provisional reporting in seriously ill children when the diagnosis indicated changes in management that could improve outcome, and where a delay in reporting until confirmation of results by Sanger sequencing could result in avoidable morbidity or mortality. In our previous experience, provisional diagnoses were reported in 17% (114 of 684) of genome sequencing cases, with a mean time to report of 3.6 days. Presentations in which 20-hour diagnoses were likely to be associated with improved outcomes included neonatal epileptic encephalopathies, metabolic diseases (as in patient 352), septic shock possibly associated with immunodeficiency (as in patient 7052), organ failure, and when extra-corporeal membrane oxygenation is considered in the absence of a known disease etiology. Thus, a circumscribed application of an autonomous diagnostic system is to identify provisional diagnoses for laboratory director review, earlier than standard rapid testing, in a subset of neonatal and pediatric ICU admissions in which morbidity or mortality is likely to be avoided by early institution of targeted treatment. It will be important to evaluate the proportion of seriously ill patients and extent of urgent healthcare settings in which a 20-hour diagnosis would inform acute interventions and for which a longer time to result would not be effective.

This disclosure demonstrated the automated extraction of a deep, digital phenome from the EHR. The analytic performance of the extraction of phenotypic features from the EHRs of children with genetic diseases by CNLP herein was considerably better than prior reports, and appeared adequate for replacement of expert manual EHR review. CNLP extracted 27-fold more phenotypic features from the EHR than those selected by experts during manual interpretation, consistent with prior reports. In addition, the mean information content of the CNLP phenome was greater than that of the phenotypic features selected by experts during manual interpretation. The superiority of deep CNLP phenomes was shown by substantially greater overlap with the expected (OMIM™) clinical features than by those selected by experts during manual interpretation. Phenotypic features selected by experts during manual interpretation had poorer diagnostic utility than CNLP-based phenotypic features when used in the autonomous diagnostic system. This concurred with two recent reports of genomic sequencing of cohorts of patients in which the rate of diagnosis was greater when more than fifteen phenotypic features were used at time of interpretation that when one to five were used.

Herein the inventors described fully automated interpretation of sequencing results. In 95 seriously ill children, the autonomous system had 97% recall and 99% precision in recapitulating 97 genetic disease diagnoses made by a team of experts. Where the system suggested more than one diagnosis, the median rank of a variant associated with the correct diagnosis was first. The three false negative autonomous results had explanations that either can be addressed by parameter adjustments or were of types that cause assessments of variant pathogenicity to vary between laboratories. Prospectively, molecular laboratory directors determined that the autonomous system made correct provisional diagnoses in three of seven seriously ill ICU infants (100% precision and recall) with an average time saving of 22:19 hours. In light of insufficient expert analysts, molecular laboratory directors, medical geneticists and genetic counselors to expand genomic diagnosis to regional ICU infants worldwide, such diagnostic performance was sufficient to suggest several, high throughput clinical applications. Supervised autonomous systems may provide effective first-tier, provisional diagnoses, allowing valuable cognitive resources to be reserved for unsolved or difficult cases, manual curation of variants, and clinical report generation which includes a summary of medical management literature. Secondly, in the roughly 67% of cases where manual interpretation fails to provide a diagnosis, it is difficult to know when analysis should be considered complete. With further development, autonomous diagnostic systems could provide an independent, objective analysis in such cases. Thirdly, autonomous systems could re-analyze unsolved cases periodically. This is burdensome to perform manually since 250 new gene-disease associations and 9,200 new variant-disease associations are reported annually. However, re-analysis yields up to 8-10% new diagnoses per annum. Automated re-analysis could include updated CNLP of the EHR, which would useful when the phenotype evolves with time. A known risk of genetic testing is over-treatment as a result of over-diagnosis. Periodic, autonomous re-analysis would also detect cases where the diagnosis is changed as a result of reclassification of the causality of the gene or pathogenicity of the variant and/or phenome overlap was minimal. An autonomous system, akin to an autopilot, can decrease the labor intensity of genome interpretation. 106 years after the invention of the autopilot, however, two pilots are still employed in cockpits of commercial aircraft. Likewise, a skilled team will still be required to curate the literature and make tough decisions/classifications for the foreseeable future.

The autonomous system has several limitations. Firstly, system performance is partly predicated on the quality of the history and physical examination, and completeness of the write-up in EHR notes. The performance of the autonomous diagnostic system, though acceptable, is anticipated to improve with additional training, increased mapping of human phenotype ontology terms associated with genetic diseases in OMIM™, Orphanet™ and the literature to SNOMED-CT™, the native language of the CNLP, inclusion of phenotypes from structured EHR fields, measurements of phenotype severity (such as phenotype term frequency in EHR documents), and material negative phenotypes (pathognomonic phenotypes whose absence rules out a specific diagnosis). As part of this, a quantitative data model is needed for improved multivariate matching of non-independent phenotypes that appropriately weights related, inexact phenotype matches. Although possible, the autonomous system did not take advantage of commercial variant database annotations, such as the Human Gene Mutation Database™, and does not eliminate the labor-intensive literature curation which is the current standard for variant reporting. Diagnosis of genetic diseases due to structural variants requires standard library preparation and additional software steps that add several hours to turnaround time. Because the autonomous system utilizes the same knowledge of allele and disease frequencies as manual interpretation, which under-represent minority races or ethnicities, pathogenicity assertions in the latter groups are less certain. Likewise, as the autonomous system utilizes the same consensus guidelines for variant pathogenicity determination as manual interpretation, it is subject to the same general limitations of assertions of pathogenicity.

The major barriers to widespread adoption of genomic medicine for seriously ill infants with disorders of unknown etiology are an untrained medical workforce and substantial shortage of domain experts, including medical geneticists, molecular laboratory directors and genetic counselors. Manual genome analysis and interpretation are very labor intensive. In addition, the extreme number of rare genetic diseases precludes easy domain mastery by non-experts. Thus, pediatric genomic medicine may be one of the first clinical areas where artificial intelligence is necessary for its general adoption. Diagnosis of seriously ill infants with diseases of unknown etiology represents an early application of autonomous diagnostic systems as such cases are abundant in ICUs and a faster time to result is critical for optimal outcomes.

### Figure Legends

FIG. 1. Flow diagrams of the diagnosis of genetic diseases by standard and rapid genome sequencing. A. Steps in conventional clinical diagnosis of a single patient by genome sequencing (GS) with manual analysis and interpretation in a minimum of 26 hours, but with mean time-to-diagnosis of sixteen days (8, 16-30). Genome sequencing was requested manually. The inventors extracted genomic DNA manually from blood, assessed DNA quality (QA), and normalized the DNA concentration manually. The inventors then manually prepared TruSeq PCR-free DNA™ sequencing libraries, performed QA again, and normalized the library concentration manually. Genome sequencing was performed on the HiSeq™ 2500 system (Illumina) in rapid run mode (RRM). Sequences were manually transferred to the DRAGEN™ Platform version 1 (Illumina) for alignment and variant calling. Phenotypic features were identified by manual review of the electronic health record (EHR). Variant files and phenotypic features were loaded manually into Opal™ software (Fabric), and interpretation was performed manually. B. Steps in autonomous diagnosis of up to six patients concurrently in a minimum of 19 hours (FIG. 6). Steps included: 1. Automation of order entry from the EHR with a portal; 2. Manual or robotic preparation of Nextera DNA Flex™ sequencing libraries directly from blood in 2.5 hours; 3. Rapid 40-fold coverage genome sequencing in 15.5 hours with the NovaSeq 6000 system and S1 flowcell (Illumina); 4. Automation of sequence transfer, alignment and variant calling in one hour with the DRAGEN platform, version 2 (Illumina); 5. Automated extraction of patient phenomes from the EHR by clinical natural language processing (CNLP), and translation to human phenotype ontology (HPO) terms in 20 seconds; 6. Automated transfer of variant and phenotype files, and automated Bayesian comparison of the CNLP phenome with those of all genetic diseases (MOON, Diploid), combined with automated assessment of the pathogenicity of their genomic variants based on aggregated literature knowledge and in silico predictive tools (InterVar) and automated display of the highest ranked provisional diagnosis(es).

FIG. 2. Clinical natural language processing can extract a more detailed phenome than manual EHR review or OMIM™ clinical synopsis. A. Example CNLP of a sentence from the EHR of an eight-day-old baby (patient 341) with maple syrup urine disease, showing four extracted HPO terms. B. Hierarchical display of HPO phenotypic features extracted by manual review of the EHR of neonate 341, CNLP (red), and expected phenotypic features (from the OMIM™ Clinical Synopsis, blue). Yellow circles: Phenotypic features extracted by both CNLP and expert review. Purple circles: Phenotypic overlap between CNLP and OMIM™. Grey circles: The location of parent terms of identified phenotypic features within the HPO hierarchy. The Information Content (IC) was defined by IC (phenotype)=−log (p_{phenotype}), where p_{phenotype} was the probability of observing the exact term or one of its subclasses across all diseases in OMIM™. Information content increases from top (general) to bottom (specific).

FIG. 3. Comparison of observed and expected phenotypic features of 375 children with suspected genetic diseases. A-D: 101 children diagnosed with 105 genetic diseases. E-H: 274 children with suspected genetic diseases that were not diagnosed by genomic sequencing. Phenotypic features identified by manual EHR review are in yellow, those identified by CNLP are in red, and the expected phenotypic features, derived from the OMIM™ Clinical Synopsis, are in blue. A. Frequency distribution of the number of phenotypic features (log-transformed) in 101 children with genetic diseases. The mean number of features detected per patient was 4.2 (SD 2.6, range 1-16) for manual review, 116.1 (SD 93.6, range 13-521) for CNLP, and 27.3 (SD 22.8, range 1-100) for OMIM™ (OMIM™ vs Manual: P<0.0001; CNLP vs OMIM™: P<0.0001; CNLP vs Manual: P<0.0001; paired Wilcoxon tests). B. Frequency distribution of information content (IC) for each phenotypic feature set in 101 diagnosed patients. The mean IC was 7.8 (SD 2.0, range 2.1-11.4) for manual review, 8.1 (SD 2.0, range 2.6-11.4) for CNLP, and 7.3 (SD 1.7, range 3.2-11.4) for OMIM™ (Manual vs OMIM™: P<0.0001; CNLP vs OMIM™:P<0.0001; Manual vs CNLP: P=0.003; Mann-Whitney U tests). C. Correlation of the mean information content of phenotypic terms with the number of phenotypic terms in each patient. Spearman's rank correlation coefficient (rₛ) was 0.24 for manually extracted phenotypic features (P=0.02), 0.44 for CNLP (P<0.0001) and −0.001 for OMIM™ (P>0.05). D. Venn diagram showing overlap of phenotypic terms by the three methods for diagnosed patients. Phenotypic features extracted by CNLP overlapped expected OMIM™ phenotypic features (mean 4.31 terms, SD 4.59, range 0-32) significantly more than manually (mean 0.92 terms, SD 1.02, range 0-4; P<0.0001, paired Wilcoxon test for the difference in the number of terms that overlap with OMIM™). E. Frequency distribution of the number of phenotypic features (log-transformed) in 274 children with suspected genetic diseases that were not diagnosed by genomic sequencing. The mean number of features was 3.0 (SD 1.9, range 1-12) for manual review and 90.7 (SD 81.1, range 6-482) for CNLP (CNLP vs Manual: P<0.0001, paired Wilcoxon test). F. Frequency distribution IC for each phenotypic feature set in 273 undiagnosed patients. The mean IC was 7.7 (SD 2.1, range 2.1-11.4) for manual review and 8.1 (SD 2.0, range 2.6-11.4) for CNLP (Manual-CNLP: P<0.0001, Mann-Whitney U test). G. Correlation of the mean information content of phenotypic terms with the number of phenotypic terms in each patient. rₛ was 0.02 for manually extracted phenotypic features (P>0.05) and 0.30 for CNLP (P<0.0001). H. Venn diagram showing overlap of phenotypic terms for undiagnosed patients by CNLP and manual methods.

FIG. 4. Venn diagram showing overlap of observed and expected patient phenotypic features in 95 children diagnosed with 97 genetic diseases. Phenotypic features identified by expert manual EHR review during interpretation are shown in yellow. Phenotypic features identified by CNLP are shown in red. The expected phenotypic features are derived from the OMIM™ Clinical Synopsis and are shown in blue. The inventors excluded eight diagnoses that were considered to be incidental findings. Phenotypes extracted by CNLP overlapped expected OMIM™ phenotypes (mean 4.55, SD 4.62, range 0-32) more than phenotypes that were manually extracted (mean 0.97, SD 1.03, range 0-4).

FIG. 5. Precision, recall, and F1-score of phenotypic features identified manually, by CNLP, and OMIM™. Data are from 101 children with 105 genetic diseases. Precision (PPV) was given by tp/tp+fp, where tp were true positives and fp were false positives. Recall (sensitivity) was given by tp/tp+fn, where fn were false negatives. A. Precision and recall calculated based on exact phenotypic feature matches. Manual vs OMIM™-Precision: mean 0.25, SD 0.30, range 0-1; Recall: mean 0.04, SD 0.06, range 0-0.25; F1: mean 0.07, SD 0.09, range 0-0.40. cNLP vs OMIM™-Precision: mean 0.04, SD 0.03, range 0-0.15; Recall: mean 0.20, SD 0.16, range 0-0.67; F₁: mean 0.06, SD 0.05, range 0-0.23. Manual vs cNLP-Precision: mean 0.71, SD 0.28, range 0-1; Recall: mean 0.03, SD 0.02, range 0-0.1; F₁: mean 0.06, SD 0.04, range 0-0.17. B. Precision and recall calculated allowing for inexact phenotype matches (terms with one degree of hierarchical separation). Manual vs OMIM™-Precision: mean 0.4, SD 0.34, range 0-1; Recall: mean 0.09, SD 0.13, range 0-1; F1: mean 0.13, SD 0.13, range 0-0.57. cNLP vs OMIM™-Precision: mean 0.09, SD 0.07, range 0-0.38; Recall: mean 0.29, SD 0.22, range 0-1; F1: mean 0.12, SD 0.08, range 0-0.38. Manual vs cNLP-Precision: mean 0.79, SD 0.24, range 0-1; Recall: mean 0.06, SD 0.04, range 0-0.19; F1: mean 0.11, SD 0.07, range 0-0.32.

FIG. 6. Flow diagram of the software components of the autonomous system for provisional diagnosis of genetic diseases by rapid genome sequencing. Abbreviations: GS: rapid whole genome sequencing; GEMS: Genome management system; HPO: Human Phenotype Ontology; LIMS: Clarity laboratory information management system. Data types were as follows: *: HL7/FHIR; †: JSON; ‡: bcl; □: vcf.

### Supplementary Materials (Example 1)

### Tables

TABLE 1
Duration and metrics for the major steps in the diagnosis of genetic diseases by genome sequencing using rapid standard methods (Std.) and a rapid, autonomous platform
(Auto.). Primary (1⁰) and secondary (2⁰) Analysis: conversion of raw data from base call to FASTQ format, read alignment to the reference genomes and variant calling. Tertiary (3⁰)
Analysis Processing: Time to process variants and phenotypic features and make them available for manual interpretation in Opal interpretation software (Fabric Genomics) or to
display a provisional, automated diagnosis(es) in MOON interpretation software (Diploid).

| | Use Type | | | | | | | | | | | | | | | | | |
|---|---|---|---|---|---|---|---|---|---|---|---|---|---|---|---|---|---|---|
| | Retrospective Patients | | | | Prospective Patients | | | | | | | | | | | | | |
| | Subject ID | | | | | | | | | | | | | | | | | |
| | 263 | | 6124 | 3003 | 6194 | | 290 | | 352 | | 362 | | 374 | | 7052 | | 412 | |
| Age | 8 days | | 14 years | 1 year | 5 days | | 3 days | | 7 weeks | | 4 weeks | | 2 days | | 17 months | | 3 days | |
| Sex | ♀ | | ♂ | ♀ | ♀ | | ♂ | | ♀ | | ♂ | | ♂ | | ♂ | | ♂ | |
| Abbreviated | Neonatal seizures | | Rhabdo- | Dystonia, | Hypoglycemia, | | Pulmonary PPHN | | Diabetic | | Neonatal | | HIE, anemia | | Pseudomonal shock | | Neonatal | |
| Presentation | | | myolysis | Dev. delay | seizures | | hemorrhage, | | ketoacidosis | | seizures | | | | septic | | seizures | |
| Method | Auto. | Auto. | Auto. | Auto. | Auto. | Std. | Auto. | Std. | Auto. | Std. | Auto. | Std. | Auto. | Std. | Auto. | Std. | Auto. | Std. |
| Number of | 51 | | 115 | 148 | 14 | 2 | 257 | 4 | 103 | 4 | 65 | 1 | 112 | 6 | 124 | 3 | 33 | 1 |
| Phenotypic | | | | | | | | | | | | | | | | | | |
| Features | | | | | | | | | | | | | | | | | | |
| Molecular | Early | | Glycogen | Dopa- | None | None | None | None | Permanent neonatal | | None | None | None | None | agamma- | | Benign | familiar |
| Diagnosis | Infantile | | Storage | Disease- | | | | | diabetes mellitus | | | | | | X-linked | | neonatal | seizures |
| | Epileptic | | | Responsive | | | | | | | | | | | globulinemia 1 | | 1 | |
| | Encephalopathy | | | Dystonia | | | | | | | | | | | | | | |
| | 7V | | | | | | | | | | | | | | | | | |
| Gene and | KCNQ2 | | PYGM | TH c.785C > G | n.a. | n.a. | n.a. | n.a. | INS | | n.a. | n.a. | n.a. | n.a. | BTK | | KCNQ2 | |
| Causative | c.727C > G | | c.2262delA | c.541C > T | | | | | c.26C > G | | | | | | c.974 + 2T > C | | c.1051C > G | |
| Variant(s) | | | c.1726C > T | | | | | | | | | | | | | | | |
| Sample/Library | 3:20 | 2:55 | 2:24 | 2:22 | 2:10 | 23:54 | 2:12 | 22:05 | 2:13 | 15:42 | 2:31 | 18:30 | 3:30 | 10:10 | 4:30 | 12:10 | 3:05 | 23:50 |
| Prep (hours) | | | | | | | | | | | | | | | | | | |
| NovaSeq Loading | 10:20 | 0:17 | 0:16 | 0:20 | 1:38* | 0:20 | 0:29 | 0:22 | 0:30 | 0:53 | 0:15 | 2:30 | 0:45 | 0:35 | 1:00 | 1:00 | 0:20 | 0:53 |
| (hours) | | | | | | | | | | | | | | | | | | |
| 2 × 101 nt | 15:36 | 15:31 | 15:34 | 15:27 | 15:26 | 24:13 | 15:25 | 24:08 | 15:21 | 22:44 | 15:17 | 33:36 | 15:17 | 21:07 | 15:19 | 22:46 | 15:58 | 21:00 |
| Sequencing | | | | | | | | | | | | | | | | | | |
| (hours) | | | | | | | | | | | | | | | | | | |
| 1⁰ & 2⁰ Analysis | 1:03 | 1:02 | 0:59 | 0:59 | 1:07 | 3:05 | 1:00 | 1:57 | 1:01 | 2:30 | 1:02 | 2:30 | 1:02 | 2:30 | 1:09 | 2:25 | 1:24 | 2:24 |
| (hours) | | | | | | | | | | | | | | | | | | |
| 3⁰ Analysis | 0:06 | 0:05 | 0:07 | 0:05 | 0:06 | 0:15 | 0:08 | 0:14 | 0:06 | 0:15 | 0:05 | 0:15 | 10:28† | 0:16 | 0:06 | 0:16 | 0:06 | 0:16 |
| Processing | | | | | | | | | | | | | | | | | | |
| (hours) | | | | | | | | | | | | | | | | | | |
| Total (hours) | 20:25 | 19:56 | 19:20 | 19:14 | 20:42* | 56:03 | 19:29 | 48:46 | 19:11 | 42:04 | 19:10 | 57:21 | 31:02† | 34:38 | 22:04 | 38:37 | 20:53 | 48:23 |

Dev. Delay: global developmental delay.
PPHN: Persistent pulmonary hypertension of the newborn.
HIE: Hypoxic ischemic encephalopathy.
n.a .: not applicable.
* Included time to thaw a second set of NovaSeq reagents.
†Included 10:20 hours of downtime, with manual restarting of the job, due to data center relocation.
Patients 263, 6124 and 3003 were retrospectively analyzed by the autonomous system. Patient 263 was analyzed two times by the autonomous system. Patients 6194, 290, 352, 362, 412, and 7072 were prospectively analyzed by both autonomous and standard diagnostic methods.

TABLE 2
Comparison of the analytic performance of standard and new library preparation, and standard and rapid genome sequencing
in retrospective samples. The standard library preparation and genome sequencing methods were TruSeq ™ PCR-free library preparation and
2 × 100 nt sequencing on a NovaSeq ™ 6000 with S2 flow cell, respectively. The new library preparation and genome sequencing methods were
Nextera Flex ™ library preparation and 2 × 100 nt sequencing on a NovaSeq ™ 6000 with S1 flow cell, respectively. The “Median” column is
the median of runs R17AA978, R17AA978, R17AA059, and R17AA119. Controls 1 and 2 are mean values for five and fifty-two samples,
respectively. Analytic performance of variant calls was assessed in sample NA12878, with comparison to the NIST Genome-in-a-bottle results
(76). Note: The NA12878 control run with the S1 flowcell and TruSeq ™ PCR free library (far right) was 2 × 151 nt.

| | Run | | | | | | | | |
|---|---|---|---|---|---|---|---|---|---|
| | R17AA978 | R17AA978 | R17AA059 | R17AA119 | Median | NA12878 | Control 1 | Control 2 | NA12878 |
| | NovaSeq ™ 6000 Flowcell | | | | | | | | |
| | S1 | | | | S1 | | S2 | S2 | S1 |
| | Library Preparation Method | | | | | | | | |
| | Nextera TM Flex | | | | xNextera ™ | | Nextera ™ | TruSeq ™ | PCR-free |
| | | | | | | | Flex | | |
| | Sample | | | | | | | | |
| | | | | | 263 × 2, | | | | |
| | 263 | 263 | 6124 | 3003 | 6124, 3003 | 1 sample | 5 samples | 52 samples | 1 sample |
| Raw Yield Per Flowcell (Gb) | 416 | 419 | 404 | 432 | 418 | 435 | 933 | 897 | 537 |
| % Reads Q > 30 | 92.00% | 92.07% | 92.11% | 94.84% | 92.09% | 90.69% | 91.50% | 91.70% | 91.96% |
| Trimmed Yield (Gb) | 153.9 | 158.9 | 165.0 | 160.7 | 159.8 | 148.9 | 183.3 | 152.8 | 164.5 |
| % Reads Mapped | 97.9% | 97.9% | 98.1% | 96.9% | 97.9% | 98.9% | 98.6% | 98.7% | 98.8% |
| % Duplicate Reads | 9.3% | 10.4% | 7.6% | 19.1% | 9.8% | 8.50% | 11.4% | 6.3% | 17.2% |
| Mean Insert Size (nt) | 386.0 | 348.0 | 336.0 | 274.0 | 342.0 | 345.1 | 315.1 | 423.4 | 514.6 |
| Average genome coverage | 42.0 | 43.0 | 44.4 | 39.0 | 42.5 | 47.5 | 49.4 | 43.6 | 32.9 |
| % OMIM genes with 100% coverage | 96.0% | 95.7% | 94.9% | 65.1% | 95.3% | 95.8% | 96.8% | 97.7% | 98.00% |
| at ≥10X | | | | | | | | | |
| Variants | 4,910,055 | 4,915,843 | 4,847,506 | 4,655,831 | 4,878,781 | 4,733,000 | 4,976,974 | 4,922,188 | 4,747,231 |
| Variants passing QC | 96.0% | 96.1% | 96.6% | 96.8% | 96.3% | 96.8% | 98.1% | 98.4% | 98.5% |
| CD Variants | 0.53% | 0.53% | 0.55% | 0.54% | 0.53% | 0.58% | 0.53% | 0.53% | 0.58% |
| Indels | 17.8% | 17.9% | 18.0% | 17.5% | 17.8% | 17.5% | 18.6% | 18.8% | 19.4% |
| CD Homozygous/Heterozygous Variant | 0.59 | 0.59 | 0.57 | 0.60 | 0.59 | 0.60 | 0.56 | 0.59 | 0.60 |
| Ratio | | | | | | | | | |
| Ti/Tv ratio | 2.02 | 2.02 | 2.02 | 2.03 | 2.02 | 2.02 | 2.02 | 2.02 | 2.01 |
| CD Ti/Tv ratio | 2.85 | 2.87 | 2.88 | 2.94 | 2.88 | 2.81 | 2.85 | 2.85 | 2.82 |
| Analytic Performance | | | | | | | | | |
| PPV (SNV) | n.a. | n.a. | n.a. | n.a | n.a. | 99.8% | 99.8% | 99.9% | 99.9% |
| PPV (indels) | n.a. | n.a. | n.a. | n.a. | n.a. | 99.0% | 97.0% | 99.3% | 99.7% |
| Sensitivity (SNV) | n.a. | n.a. | n.a. | n.a. | n.a. | 99.7% | 99.6% | 99.7% | 99.8% |
| Sensitivity (indels) | n.a. | n.a. | n.a. | n.a. | n.a. | 95.5% | 96.3% | 99.0% | 99.4% |

Abbreviations: nt: Nucleotides;
FC: flowcell;
Gb: gigabase;
Q: Quality score;
OMIM: Online Mendelian Inheritance in Man;
QC: Quality Control;
CD: Coding Domain;
Ti/Tv ratio: ratio of the number of nucleotide transitions to the number of nucleotide transversions;
PPV: Positive predictive value;
SNV: single nucleotide variants;
indels: nucleotide insertion-deletion variants.

TABLE 3
Comparison of the analytic performance of standard and new library preparation and genome sequencing methods
in seven matched prospective samples. The standard library preparation and genome sequencing methods were
TruSeq ™ PCR-free library preparation and NovaSeq ™ 6000 with S2 flow cell, respectively, with the exception of subjects
7052 and 412, where the library preparation was done with the KAPA Hyper ™ kit. The new library preparation and genome
sequencing methods were Nextera ™ Flex library preparation and NovaSeq ™ 6000 with S1 flow cell, respectively.

| Run | R18AA202 | Std. | R18AA218 | Std. | R18AA922 | Std | R18AB113 | Std |
|---|---|---|---|---|---|---|---|---|
| Subject | 6194 (Prospective) | | 290 (Prospective) | | 352 (Prospective) | | 362 (Prospective) | |
| Library Prep | Nextera | TruSeq | Nextera | TruSeq | Nextera | TruSeq | Nextera | TruSeq |
| Method | | | | | | | | |
| Flow cell | S1 | S2 | S1 | S2 | S1 | S2 | S1 | S2 |
| Raw Yield Per | 389.9 | 945.4 | 381.8 | 946 | 365.3 | 869.9 | 398.3 | 440.7 |
| Flow cell (Gb) | | | | | | | | |
| Reads Q >=30 | 90.90% | 93.70% | 91.30% | 93.10% | 89.80% | 90.70% | 92.20% | 90.00% |
| % Cluster passing | 69.8/82.9 | 82.1/82.0 | 73.9/75.6 | 82.2/82.0 | 73.8/69.3 | 75.5/75.5 | 78.9/77.1 | 36.7/39.9 |
| filter, L1/L2 | | | | | | | | |
| % Error rate | 0.19/0.42 | 0.27/0.47 | 0.25/0.65 | 0.27/0.37 | 0.25/0.45 | 0.31/0.37 | 0.20/0.36 | 0.33/0.41 |
| (ΦX174), R1/R2 | | | | | | | | |
| Trimmed Yield | 174.1 | 172.3 | 168.6 | 218.2 | 141 | 144.2 | 164.3 | 148.4 |
| (Gb) | | | | | | | | |
| Reads Mapped | 97.70% | 98.60% | 97.30% | 98.30% | 97.20% | 98.60% | 97.40% | 98.50% |
| Duplicate Reads | 11.50% | 6.50% | 11.60% | 7.30% | 8.90% | 9.20% | 9.90% | 3.90% |
| Mean Insert Size | 361.2 | 405.8 | 223.7 | 430 | 373.4 | 419.8 | 369 | 410 |
| (nt) | | | | | | | | |
| Average genome | 44.8 | 48.4 | 54 | 60.4 | 39.1 | 39.3 | 43.1 | 42.8 |
| coverage | | | | | | | | |
| % OMIM genes | 95.80% | 97.90% | 93.30% | 98.20% | 95.80% | 97.80% | 95.70% | 96.60% |
| w. >10X × | | | | | | | | |
| 100% nt | | | | | | | | |
| Variants | 4,687,590 | 4,881,456 | 4,776,648 | 5,016,422 | 4,765,467 | 4,934,554 | 4,719,091 | 4,917,044 |
| Variants passing | 96.90% | 98.30% | 97.00% | 98.20% | 97.00% | 98.60% | 97.00% | 98.20% |
| QC | | | | | | | | |
| CD Variants | 0.57% | 0.52% | 0.57% | 0.53% | 0.54% | 0.56% | 0.55% | 0.54% |
| Indels | 18.20% | 18.90% | 18.00% | 18.90% | 18.00% | 18.60% | 17.70% | 18.50% |
| Ti/Tv ratio | 2.02 | 2.02 | 2.03 | 2.03 | 2.02 | 2.03 | 2.02 | 2.01 |

| Run | R18AB229 | Std | R18AB352 | Std | R18AB672 | Std |
|---|---|---|---|---|---|---|
| Subject | 374 (Prospective) | | 7052 (Prospective) | | 412 (Prospective) | |
| Library Prep | Nextera | KAPA | Nextera | KAPA | Nextera | KAPA |
| Method | | Hyper | | Hyper | | Hyper |
| Flow cell | S1 | S2 | S1 | S2 | S1 | S2 |
| Raw Yield Per | 420.8 | 899.1 | 383.4 | 860.2 | 422.1 | 908.2 |
| Flow cell (Gb) | | | | | | |
| Reads Q >=30 | 93.30% | 91.60% | 90.10% | 90.10% | 92.90% | 91.60% |
| % Cluster passingx | 83.0/81.8 | 78.3/77.8 | 75.49/74.7 | 75.2/74.1 | 83.1/82.3 | 78.9/78.8 |
| filter, L1/L2 | | | | | | |
| % Error rate | 0.20/0.40 | 0.25/0.35 | 0.26/0.50 | 0.31/0.36 | 0.22/0.32 | 0.28/0.29 |
| (ΦX174), R1/R2 | | | | | | |
| Trimmed Yield | 185.5 | 267.8 | 156.4 | 138 | 183.4 | 203 |
| (Gb) | | | | | | |
| Reads Mapped | 98.00% | 98.50% | 97.30% | 98.30% | 98.60% | 98.60% |
| Duplicate Reads | 11.70% | 14.60% | 8.30% | 9.40% | 14.00% | 13.40% |
| Mean Insert Size | 266.9 | 423.8 | 371.4 | 428.4 | 338.1 | 416.2 |
| (nt) | | | | | | |
| Average genome | 48 | 68.4 | 41.6 | 37.3 | 47.6 | 50.9 |
| coverage | | | | | | |
| % OMIM genes | 96.00% | 98.40% | 95.20% | 97.80% | 96.90% | 98.20% |
| w. >10X × | | | | | | |
| 100% nt | | | | | | |
| Variants | 4,758,713 | 5,001,708 | 4,821,433 | 4,981,748 | 4,958,194 | 4,965,915 |
| Variants passing | 98.10% | 98.00% | 98.10% | 98.60% | 98.10% | 98.20% |
| QC | | | | | | |
| CD Variants | 0.55% | 0.53% | 0.56% | 0.53% | 0.56% | 0.53% |
| Indels | 19.60% | 18.80% | 17.60% | 18.50% | 18.70% | 18.90% |
| Ti/Tv ratio | 2.01 | 2.01 | 2.03 | 2.02 | 2.01 | 2.02 |

Abbreviations: L: lane;
R: read; nt:
Nucleotides;
Gb: gigabase;
Q: Quality score;
OMIM: Online Mendelian Inheritance in Man;
QC: Quality Control;
CD: Coding Domain;
Ti/Tv ratio: ratio of the number of nucleotide transitions to the number of nucleotide transversions.

TABLE 4
Characteristics of sixteen children with genetic diseases used to train CNLP.

| | | | | | | | | | | | | Age at | | |
|---|---|---|---|---|---|---|---|---|---|---|---|---|---|---|
| | | | | | | | | | | | | enroll- | | |
| | | rWES or | | Affected | OMIM | | de novo or | | | V1 | V2 | ment | | Consang- |
| Famiy | S, D, T | rWGS ® | Disease | Gene | ID | Inheritance | inherited | Variant 1 (V1) | Variant 2 (Vw) | P/LP | P/LP | (days) | Sex | unity |
| 6007 | T | rWGS ® | EIEE9 | PCDH19 | 300088 | AD | DN | Xq22del | | | | 423 | F | No |
| 6008 | S | rWGS ® | Glioblastoma | BRCA1 | 604370 | AD | n.d. | c.5159G > A, p.Arg1720Gln | | | | 4563 | F | No |
| | | | | | | | | c.3096_3100delCAAAG; | | | | | | |
| 6012 | S | rWGS ® | Coffin-Siris syndrome 1 | ARID1B | 135900 | AD | DN | p.Lys1033ArgfsTer32 | | | | 231 | F | No |
| 6014 | S | rWGS ® | Nemaline myopathy 2 | NEB | 256030 | AR | n.d. | c.19262 + 1G > A | c.2416-1G > C | | | 35 | M | No |
| 6024 | T | rWGS ® | Hypophosphatemic | PHEX | 307800 | XLD | I | c.1604C > T,p.Thr535Met | | | | | | |
| | | | rickets, X-linked | | | | | | | | | | | |
| | | | dominant | | | | | | | | | 137 | M | No |
| 6026 | T | rWGS ® | Alagille syndrome 1 | 20p12.2 del | 118450 | AD | DN | Chr20: 10471400- | | | | 80 | M | U |
| | | | | | | | | 13459331del | | | | | | |
| 6030 | T | rWGS ® | Neurofibromatosis 1; | NF1 & | 162200, | AD, AD | DN, I | c.5118delT; | c.3184delG | LP | LP | 227 | M | No |
| | | | Left ventricular | MYBPC3 | 615396 | | | p.Val1707PhefsTer | p.Val1062LeufsTer13 | | | | | |
| | | | noncompaction 10 | | | | | | | | | | | |
| 6030 | T | rWGS ® | Catecholaminergic | RYR2 | 604772 | AD | DN | C.646C > T; | | | | 6087 | F | No |
| | | | polymorphic- | | 615396 | | | p.Ala549Val | | | | | | |
| | | | Ventricular | | | | | | | | | | | |
| | | | tachycardia 1 | | | | | | | | | | | |
| 6037 | T | rWGS ® | Neonatal cholestasis; | none | none | n.a. | n.a. | n.a. | | | | 60 | M | U |
| | | | Extrahepatic | | | | | | | | | | | |
| | | | billiary atresia | | | | | | | | | | | |
| 6041 | T | rWGS ® | EIEE7 | KCNQ2 | 613720 | AD | DN | c.875T > C; | | | | 2 | F | No |
| | | | | | | | | p.Leu292Pro | | | | | | |
| 6044 | S | rWGS ® | Pieuropulmonary | DICER | 601200 | AD | n.d. | c.2771T > G; | | | | 564 | M | U |
| | | | blastoma | | | | | p.Leu924* | | | | | | |
| 6045 | S | rWGS ® | Meduloblastoma | none | none | n.a. | n.a. | n.a. | | | | 5475 | M | U |
| 6051 | S | rWGS ® | Glioma | none | none | n.a. | n.a. | n.a. | | | | 2555 | M | U |
| 6052 | T | rWGS ® | MECRCN | TANGO2 | 616878 | AR | I | c.605 + 1G > A | 33 kb del | | | 898 | F | U |
| | | | | | | | | | TANGO2 exons 3-9 | | | | | |
| 6066 | D | rWGS ® | Neonatal | none | non | n.a. | n.a. | n.a. | | | | 60 | F | U |
| | | | cholestasis; | | | | | | | | | | | |
| | | | Cleft lip and palate | | | | | | | | | | | |
| 6117 | D | rWGS ® | Neonatal | none | none | n.a. | n.a. | n.a. | | | | 60 | F | U |
| | | | cholestasis | | | | | | | | | | | |

Abbreviations: EIEE: Early Infantile Epileptic Encephalopathy;
AD: Autosomal Dominant;
DN: de novo;
P: Pathogenic;
LP: Likely Pathogenic;
M: Male;
F: Female;
S: Singleton;
D: Duo;
T: Trio;
I: Inherited;
XLD: X-linked dominant;
MECRN: Metabolic encephalomyopathic crises, recurrent, with rhabdomyolysis, cardiac arrhythmias, and neurodegeneration;
U: undetermined;
OMIM: Online Mendelian Inheritance in Man.

TABLE 5
Precision and recall phenotypic features extracted by CNLP from EHRs in ten children with genetic diseases. Precision = tp/tp + fp. Recall = tp/tp + fn.

| | | | | | | | | | | | | Age at | | | | | | |
|---|---|---|---|---|---|---|---|---|---|---|---|---|---|---|---|---|---|---|
| | | | | | | | | | | | | enroll- | | | | | | OMIN CF |
| | S, or | rWES or | | Affected | OMIM | | denovo or | Variant 1 | Variant 2 | V1 | V2 | ment | | Consang- | CNLP | CNLP | CNLP | detected |
| Family | T | rWGS ® | Disease | Gene | ID | Inheritance | inherited | (V1) | (Vw) | P/LP | P/LP | (days) | Sex | unity | Features | Prescision | Recall | by CNLP |
| 201 | T | rWES | Prader | Willi15q11- | 176270 | AD | DM | Chr15: 23684685- | | | | 3 | ♀ | U | 26 | 0.88 | n.d. | 3% |
| | | | Syndrome | q13del | | | | 26108259del | | | | | | | | | | |
| 205 | T | rWGS ® | Dursun | G6CP3 | 612541 | AR | I | c.207dupC, | C.199_218 + | P | P | 2 | ♂ | No | 96 | 0.80 | n.d. | 15% |
| | | | Syndrome | | | | | p.Ile70HisfsTer17 | 1delCTCAACC | | | | | | | | | |
| | | | | | | | | | TCATCTTC | | | | | | | | | |
| | | | | | | | | | AAGTGG | | | | | | | | | |
| 213 | S | rWGS ® | Visceral | NODAL | 270100 | AD | I | C.778G > A; | | | | 3 | ♂ | U | 95 | 0.67 | 0.91 | 56% |
| | | | Heterotaxy | | | | | p.Gly260Arg | | | | | | | | | | |
| | | | 5 | | | | | | | | | | | | | | | |
| 233 | T | rWGS ® | Tuberous | TSC1 | 191100 | AD | DN | c.1498C > T, | | | | 3 | ♀ | No | 158 | 0.51 | 0.91 | 14% |
| | | | Sclerosis 1 | | | | | p.Arg500Ter | | | | | | | | | | |
| 243 | T | rWGS ® | Pyridoxine | ALDH7A1 | 266100 | AR | I | c.32iC > T, | c.1279G > C, | | | 7 | ♂ | No | 85 | 0.82 | 0.93 | 21% |
| | | | dependent | | | | | p.Arg110Ter | p.Glu427Gln | | | | | | | | | |
| | | | seizures | | | | | | | | | | | | | | | |
| 6,094 | T | rWGS ® | Arginino- | ASL | 207900 | AR | I | c.706C > T, | c.706C > T, | P | P | 7 | ♀ | Yes | 90 | 0.83 | | 11% |
| | | | succinic | | | | | p.Art236Trp | p.Arg236Trp | | | | | | | | | |
| | | | Aciduria | | | | | | | | | | | | | | | |
| 6,098 | T | rWGS ® | Gaucher | GBA | 230800 | AR | I | c.1503C > G, | c.1448T > C, | | | 214 | ♀ | No | 96 | 0.9 | | 21% |
| | | | disease | | | | | p.Asn501Lys | p.Leu483Pro | | | | | | | | | |
| 6,108 | T | rWGS ® | Tuberous | TSC2 | 613254 | AD | DN | c.935_936delTC, | | | | 3 | ♂ | No | 83 | 0.76 | | 5% |
| | | | Sclerosis 2 | | | | | p.Leu312GlnfsTer25 | | | | | | | | | | |
| 7,003 | T | rWGS ® | EIEE6 | SCN1A | 607208 | AD | DN | c.5555T > C, | | | | 424 | ♂ | U | 44 | 0.84 | 0.93 | 25% |
| | | | | | | | | p.Met1852Thr | | | | | | | | | | |
| 7,004 | T | rWGS ® | Hyper- | MYH7 | 192600 | AD | I | c.746G > A, | | | | 5171 | ♂ | U | 71 | 0.94 | 0.96 | 44% |
| | | | trophic | | | | | p.Arg249Gln | | | | | | | | | | |
| | | | cardio- | | | | | | | | | | | | | | | |
| | | | myopathy | | | | | | | | | | | | | | | |
| | | | type 1 | | | | | | | | | | | | | | | |

| | | | | |
|---|---|---|---|---|
| Mean | 86.7 | 0.80 | 0.93 | 22% |
| Standard Deviation | 32.8 | 0.13 | 0.02 | 0.17 |

Abbreviations: EIEE: Early Infantile Epileptic Encephalopathy;
AD: Autosomal Dominant;
AR: Autosomal Recessive;
DN: de novo;
P: Pathogenic;
LP: Likely Pathogenic;
S: Singleton;
T: Trio;
I: Inherited;
U: undetermined;
OMIM: Online Mendelian Inheritance in Man;
CF:Clinical Feature.

TABLE 6
Number of structural variants shortlisted by MOON ™ and
rank of the causal variant in MOON ™ in 11 children
with genetic diseases. All samples were run as singletons.

| | rWES/ | # SV calls | # SV shortlisted | Causal SV rank |
|---|---|---|---|---|
| Family | rWGS ® | in gVCF | by MOON | in MOON |
| 201 | rWES | 6 | 2 | 1 |
| 259 | rWES | 16 | 9 | 1 |
| 286 | rWES | 7 | 3 | 1 |
| 319 | rWES | 12 | 4 | 1 |
| 217 | rWGS ® | 21 | 8 | 1 |
| 223 | rWGS ® | 16 | 9 | 5 |
| 302 | rWGS ® | 22 | 17 | 13 |
| 6140 | rWGS ® | 11 | 8 | 1 |
| 6146 | rWGS ® | 23 | 15 | 9 |
| 6164 | rWGS ® | 25 | 15 | 12 |
| 7023 | rWGS ® | 17 | 12 | 12 |

| | | | |
|---|---|---|---|
| Mean, rWES | 10.3 | 4.5 | Median rWGS ®, |
| Mean, rWGS ® | 19.3 | 12.0 | rWES 1.0 |

Abbreviations: gVCF: Genomic variant call file; rWES: rapid whole exome sequencing; rWGS ®: rapid whole genome sequencing; SV: structural variant.

TABLE 7
Summary statistics of provisional diagnoses reported for rapid clinical
genome sequencing. Total probands refers to children tested.

| | | Mean Time to |
|---|---|---|
| | | Provisional Report |
| | Provisional | (Sample Accession to |
| Total | Reports | Preliminary Results |
| Probands | Returned | Communicated), Days |
| 684 | 114 (16.7%) | 3.6 |

1. A method comprising:
a) determining a comprehensive set of genetic diseases;
b) identifying genetic diseases of the comprehensive set that are severe and have childhood onset;
c) determining efficacy and quality of evidence of efficacy of a comprehensive set of available therapeutic interventions for the genetic disease identified in (b);
d) determining a comprehensive set of genes associated with genetic diseases that have at least one available therapeutic intervention;
e) determining a comprehensive set of pathogenic or likely pathogenic genetic variants of the comprehensive set of genes determined in (d);
f) determining population frequency of the genetic variants;
g) for recessive genetic diseases of the genetic variants, determining which recessive genetic diseases occur in cis in populations;
h) analyzing results of (e), (f) and (g) to generate a revised list of pathogenic or likely pathogenic genetic variants;
i) performing genetic sequencing of a genomic DNA sample from a subject;
j) determining genetic variant diplotypes of the genomic DNA;
k) comparing the genetic variant diplotypes with the results of (h) to determine whether the subject screens positive for a genetic disease for which an effective treatment currently exists or can be developed; and
l) generating a report comprising results of any of (a)-(k).

2. The method of claim 1, further comprising: m) recalculating population allele frequencies or diplotype allele frequencies of (f) to include results of (j).

3. The method of claim 1, further comprising: n) performing confirmatory testing of results of (k) to determine whether they are true or false positive results.

4. The method of claim 3, further comprising: o) providing an available therapeutic intervention from the comprehensive set of available therapeutic interventions of (c) if the results of (n) are true positive results.

5. The method of claim 3, further comprising: p) updating variant pathogenicity assertions of (e) to include results of (n).

6. The method of claim 3, further comprising determining a fetal phenotype or newborn phenotype.

7. The method of claim 4, further comprising: q) measuring longitudinal outcomes following available therapeutic interventions.

8. The method of claim 7, further comprising: r) updating the available therapeutic interventions of (c) to include results of (q).

9. (canceled)

10. The method of claim 4, wherein the available therapeutic intervention is selected from the group consisting of gene therapy, protein replacement therapy, antisense oligonucleotide therapy and gene editing therapy.

11. (canceled)

12. The method of claim 1, wherein the genetic variants are selected from the group consisting of a single nucleotide polymorphism (SNP), deletion/insertion polymorphism (INDEL), structural variant (SV), copy number variant (CNV), loss of heterozygosity (LOH), microsatellite instability (MSI), variable number of tandem repeats (VNTR), simple sequence repeat (SSR), mobile insertion element, methylation variant, and chromosomal variant (such as aneuploidy or translocation).

13. The method of claim 1, wherein genetic sequencing comprises, genome sequencing, rapid whole genome sequencing (rapid WGS), ultra-rapid whole genome sequencing, exome sequencing, rapid whole exome sequencing (rWES) or gene panel sequencing.

14. (canceled)

15. The method of claim 14, wherein the DNA sample is from a biological sample selected from blood, dried blood spot, saliva, buccal smear/swab, or cord blood.

16. The method of claim 1, wherein genetic sequencing is performed for both biological parents and only results in which trio diplotypes fit a known inheritance pattern of a specific genetic disease are obtained.

17. The method of claim 16, wherein genetic sequencing is performed for both biological parents, and wherein parental health status (healthy or affected) is used to obtain only results in which parental diplotypes fit a known inheritance pattern of a specific genetic disease.

18. The method of claim 17, wherein genetic variants present in the subject's genome and not in the parental genome are utilized to determine a diagnosis for the subject.

19. The method of claim 1, wherein the subject is an infant, fetus or newborn.

20-21. (canceled)

22. The method of claim 4, wherein the available therapeutic intervention is selected from the group consisting of surgery, diet, drug, genetic/gene therapies, device, adjuvant chemotherapy, neoadjuvant chemotherapy, radiation therapy, hormone therapy, cytotoxic therapy, immunotherapy, adoptive T cell therapy, targeted therapy, and any combinations thereof.

23. A system comprising:
a controller including at least one processor and non-transitory memory, wherein the controller is configured to perform one or more steps of claim 1.
